(19) United States
(12) Patent Application Publication
(10) Pub. No.: US 2022/0289692 A1
(43) Pub. Date: Sep. 15, 2022

(54) NLRP3 INHIBITORS

(71) Applicant: Inflazome Limited, Dublin 2 (IE)

(72) Inventors: David MILLER, Cambridge (GB); Angus MACLEOD, Cambridge (GB); Jonathan SHANNON, Nottingham (GB); Jokin CARRILLO ARREGUI, Nottingham (GB); Diana CASTAGNA, Nottingham (GB); Jimmy VAN WILTENBURG, Groningen (GB); Jacobus Antonius Joseph DEN HARTOG, Groningen (NL)

(21) Appl. No.: 17/636,021

(22) PCT Filed: Sep. 4, 2020
(86) PCT No.: PCT/EP2020/074738

§ 371 Date: Feb. 16, 2022

(30) Foreign Application Priority Data
Sep. 6, 2019 (GB) 1912865.1
Mar. 31, 2020 (GB) 2004683.6

(51) Int. Cl. C07D 249/14; C07D 401/12; C07D 401/14

(52) U.S. Cl. CPC C07D 249/14; C07D 401/12; C07D 401/14

(57) ABSTRACT

The present invention relates to substituted₅-membered nitrogen containing heteroaryl compounds, such as triazole esters, where the heteroaryl ring is further substituted via a linking group such as —NH— with a cyclic group which in turn is substituted at the a-position. The present invention further relates to associated salts, solvates, prodrugs and pharmaceutical compositions, and to the use of such compounds in the treatment and prevention of medical disorders and diseases, most especially by NLRP₃ inhibition.

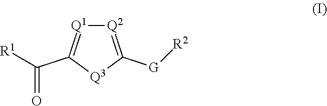

### FIELD OF THE INVENTION

The present invention relates to substituted 5-membered nitrogen containing heteroaryl compounds, such as triazole esters, where the heteroaryl ring is further substituted via a linking group such as —NH— with a cyclic group which in turn is substituted at the α-position. The present invention further relates to associated salts, solvates, prodrugs and pharmaceutical compositions, and to the use of such compounds in the treatment and prevention of medical disorders and diseases, most especially by NLRP3 inhibition.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3−/− mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al., J Pharmacol Exp Ther, 299: 187-197, 2001). CRIDs are a class of diarylsulfonylurea-containing compounds that inhibit the post-translational processing of IL-1β. Post-translational processing of IL-1β is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184623 A1, WO 2017/184624 A1, WO 2018/015445 A1, WO 2018/136890 A1, WO 2018/215818 A1, WO 2019/008025 A1, WO 2019/008029 A1, WO 2019/034686 A1, WO 2019/034688 A1, WO 2019/034690 A1, WO 2019/034692 A1, WO 2019/034693 A1, WO 2019/034696 A1, WO 2019/034697 A1, WO 2019/043610 A1, WO 2019/092170 A1, WO 2019/092171 A1, and WO 2019/092172 A1). In addition, WO 2017/184604 A1 and WO 2019/079119 A1 disclose a number of sulfonylamide-containing compounds as inhibitors of NLRP3. Certain sulfoximine-containing compounds are also disclosed as inhibitors of NLRP3 (WO 2018/225018 A1, WO 2019/023145 A1, WO 2019/023147 A1, and WO 2019/068772 A1).

A new class of NLRP3 inhibitors, encompassing substituted 5-membered nitrogen containing heteroaryl compounds such as sulfonyl triazoles, is disclosed in WO 2019/211463 A1. Further amino heterocyclic compounds are disclosed as having inflammasome inhibitory activity in WO 2020/157069 A1.

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

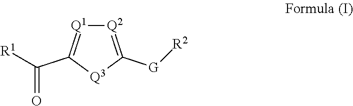

wherein:
Q¹ and Q² are each independently selected from N or CR^{q}, provided that at least one of Q¹ and Q² is N;
Q³ is O, S or NR^{qq};
each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
each R^{qq} is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
G is —O—, —C(R^{g})₂—, or —NR^{qq}—;
each R^{g} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton, or any two R^{g} may, together with the carbon atom to which they are attached, form a cyclic group wherein the cyclic group may optionally be substituted;
each R^{gg} is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
R¹ is hydrogen, —OH, —NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position, wherein R² may optionally be further substituted.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds.

Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

As used herein, where it is stated that a cyclic group is monocyclic, it is to be understood that the cyclic group is not substituted with a divalent bridging substituent (e.g. —O—, —S—, —NH—, —N(R^{β})—, —N(O)(R^{β})—, —N⁺(R^{β})₂— or —R^{α}—) so as to form a bridged, fused or spiro substituent. However, unless stated otherwise, a substituted monocyclic group may be substituted with one or more monovalent cyclic groups. Similarly, where it is stated that a group is bicyclic, it is to be understood that the cyclic group including any bridged, fused or spiro divalent bridging substituents attached to the cyclic group, but excluding any monovalent cyclic substituents, is bicyclic.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl.

Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

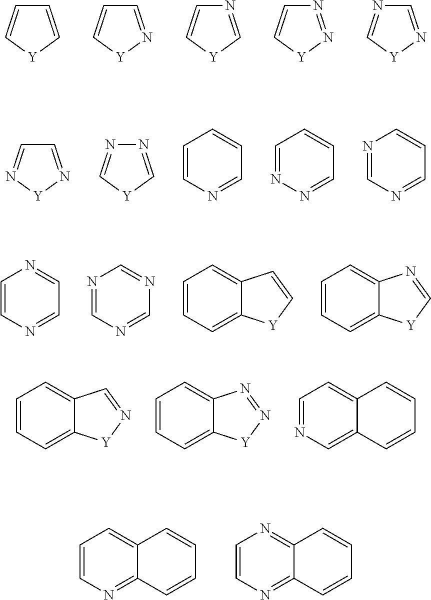

wherein Y=O, S or NH.

Unless stated otherwise, where a cyclic group or moiety is stated to be non-aromatic, such as a cycloalkyl, cycloalkenyl or non-aromatic heterocyclic group, it is to be understood that the group or moiety, excluding any ring systems which are part of or formed by substituents, is non-aromatic. Similarly, where a cyclic group or moiety is stated to be aromatic, such as an aryl or a heteroaryl group, it is to be understood that the group or moiety, excluding any ring systems which are part of or formed by substituents, is aromatic. A cyclic group or moiety is considered non-aromatic, when it does not have any tautomers that are aromatic. When a cyclic group or moiety has a tautomer that is aromatic, it is considered aromatic, even if it has tautomers that are not aromatic.

By way of example, the following are considered aromatic heterocyclic groups, because they have an aromatic tautomer:

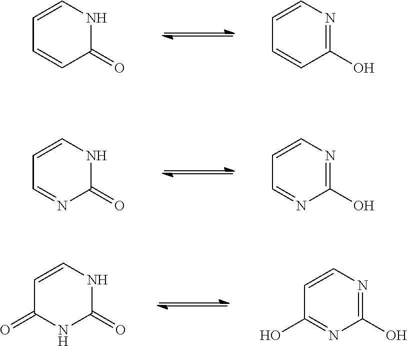 [SMILES: O=c1cccc[nH]1 ; Oc1ccccn1 ; O=c1nccc[nH]1 ; Oc1ncccn1 ; O=c1cc[nH]c(=O)[nH]1 ; Oc1ccnc(O)n1]

For the avoidance of doubt, the term “non-aromatic heterocyclic group” does not exclude heterocyclic groups or moieties which may possess aromatic character only by virtue of mesomeric charge separation. For example, the following is considered a non-aromatic heterocyclic group, because it does not have an aromatic tautomer:

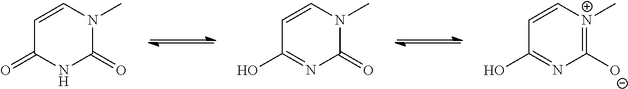

because the last shown structure is not taken into consideration because of mesomeric charge separation.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety, such as the hydrocarbyl group of R¹:

(i) each hydrogen atom may optionally be replaced by a monovalent substituent independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; R^{α}—C(═NH)NHR^{β}; —R^{α}—C(═NH)N(R^{β})₂; —R^{α}—C(═NR^{β})R^{β}; —R^{α}—C(═NR^{β})NHR^{β}; R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —NH—COOR^{β}; —NR^{β}—COOR^{β}; —NH—C(═NH)R^{β}; NR^{β}—C(═NH)R^{β}; —NH—C(═NH)NH₂; —NR^{β}—C(═NH)NH₂; —NH—C(═NH)NHR^{β}; NR^{β}—C(═NH)NHR^{β}; —NH—C(═NH)N(R^{β})₂; —NR^{β}—C(═NH)N(R^{β})₂; —NH—C(═NR^{β})R^{β}; NR^{β}—C(═NR^{β})R^{β}; —NH—C(═NR^{β})NHR^{β}; —NR^{β}—C(═NR^{β})NHR^{β}; —NH—C(═NR^{β})N(R^{β})₂; NR^{β}—C(═NR^{β})N(R^{β})₂; —NH—C(═NOH)R^{β}; —NR^{β}—C(═NOH)R; —NH—C(═NOR^{β})R^{β}; NR^{β}—C(═NOR^{β})R^{β}; —CONH₂; —CONHR^{β}; —CON(R^{β})₂; —NH—CONH₂; —NR^{β}—CONH₂; —NH—CONHR^{β}; —NR^{β}—CONHR^{β}; —NH—CON(R^{β})₂; —NR^{β}—CON(R^{β})₂; —R^{α}—NH—CHO; R^{α}—NR^{β}—CHO; —R^{α}—NH—COR^{β}; —R^{α}—NR^{β}—COR^{β}; —R^{α}—NH—COOR^{β}; —R^{α}—NR^{β}—COOR^{β}; R^{α}—NH—C(═NH)R^{β}; —R^{α}—NR^{β}—C(═NH)R^{β}; —R^{α}—NH—C(═NH)NH₂; —R^{α}—NR^{β}—C(═NH)NH₂; R^{α}—NH—C(═NH)NHR^{β}; —R^{α}—NR^{β}—C(═NH)NHR^{β}; —R^{α}—NH—C(═NH)N(R^{β})₂; R^{α}—NR^{β}—C(═NH)N(R^{β})₂; —R^{α}—NH—C(═NR^{β})R^{β}; —R^{α}—NR^{β}—C(═NR^{β})R^{β}; R^{α}—NH—C(═NR^{β})NHR^{β}; —R^{α}—NR^{β}—C(═NR^{β})NHR^{β}; —R^{α}—NH—C(═NR^{β})N(R^{β})₂; R^{α}—NR^{β}—C(═NR^{β})N(R^{β})₂; —R^{α}—NH—C(═NOH)R^{β}; —R^{α}—NR^{β}—C(═NOH)R^{β}; R^{α}—NH—C(═NOR)R^{β}; —R^{α}—NR^{β}—C(═NOR^{β})R^{β}; —R^{α}—CONH₂; —R^{α}—CONHR^{β}; —R^{α}—CON(R^{β})₂; —R^{α}—NH—CONH₂; —R^{α}—NR^{β}—CONH₂; —R^{α}—NH—CONHR^{β}; R^{α}—NR—CONHR^{β}; —R^{α}—NH—CON(R^{β})₂; —R^{α}—NR^{β}—CON(R^{β})₂; —O—R^{α}—OH; —O—R^{α}—OR⁶² ; O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon or nitrogen atom may optionally be replaced by a π-bonded substituent independently selected from oxo (═O), ═S, ═NH or ═NR^{β}; and/or

(iii) any sulfur atom may optionally be substituted with one or two π-bonded substituents independently selected from oxo (═O), ═NH or ═NR^{β}; and/or

(iv) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C≡CH, oxo (═O), phenyl, halophenyl, or optionally halo-substituted 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

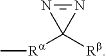

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

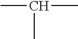

is replaced by

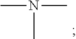

—CH₂— is replaced by —NH—, —O— or —S—;
—CH₃ is replaced by —NH₂, —OH or —SH;
—CH═ is replaced by —N═;
CH₂≡ 32 is replaced by NH═, O═ or S═; or
CH≡ is replaced by N≡;

provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

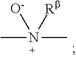

or
—CH₂— is replaced by

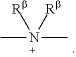

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

Unless stated otherwise, any reference to a compound or group is to be considered a reference to all tautomers of that compound or group. Thus, for example, any reference to a compound of formula (I) wherein Q¹ and Q² are both N and Q³ is NH is to be understood to encompass the tautomeric forms (a), (b) and (c) shown below:

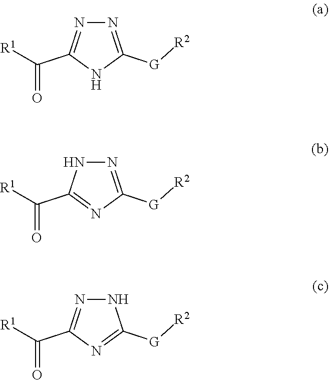

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

For the avoidance of doubt, where it is stated that a compound or a group, such as R¹, R² or L, contains from x to y atoms other than hydrogen or halogen, it is to be understood that the compound or group as a whole, including any optional substituents, contains from x to y atoms other than hydrogen or halogen. Such a compound or group may contain any number of hydrogen or halogen atoms. Similarly, where it is stated that a compound or a group, such as R¹, R² or L, contains from x to y atoms other than hydrogen, it is to be understood that the compound or group as a whole, including any optional substituents, contains from x to y atoms other than hydrogen. Such a compound or group may contain any number of hydrogen atoms.

As stated, Q¹ and Q² are each independently selected from N or CR^{q}, provided that at least one of Q¹ and Q² is N. For example, Q¹ may be N where Q² is CR^{q}, or Q¹ may be CR^{q} where Q² is N, or both Q¹ and Q² may be N. Typically, Q¹ and Q² are both N.

Where Q¹ or Q² is CR^{q}, each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton.

In one embodiment, each R^{q} where present is independently selected from hydrogen or a halo, —OH, —NH₂, —SH, or a saturated or unsaturated C₁-C₁₂ hydrocarbyl group, wherein the C₁-C₁₂ hydrocarbyl group may be straight-chained or branched, or be or include one or more cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{q} is optionally substituted, typically it is substituted with one or more groups independently selected from halo, —CN, —OH, —NH₂, —N(O)(R^{qp})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

Typically, each R^{q} where present is independently selected from hydrogen or a halo or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically, each R^{q} where present is independently selected from hydrogen or a fluoro, chloro, C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{q} where present may be independently selected from hydrogen or a fluoro, methyl, ethyl, n-propyl, isopropyl or cyclopropyl group, wherein any methyl, ethyl, n-propyl, isopropyl or cyclopropyl group may optionally be substituted with one or more fluoro groups.

Most typically, each R^{q} where present is hydrogen. In such an embodiment, Q¹ and Q² may each independently be selected from N or CH, provided that at least one of Q¹ and Q² is N.

As stated, Q³ is O, S or NR^{qq}, where each R^{qq} is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton.

In one embodiment, Q³ is O or NR^{qq}. More typically, Q³ is NR^{qq}.

In one embodiment, each R^{qq} where present is independently selected from hydrogen or a saturated or unsaturated C₁-C₁₂ hydrocarbyl group, wherein the C₁-C₁₂ hydrocarbyl group may be straight-chained or branched, or be or include one or more cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{qq} is optionally substituted, typically it is substituted with one or more groups independently selected from halo, —CN, —OH, —NH₂, —N(O)(R^{qp})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

Typically, each R^{qq} where present is independently selected from hydrogen or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically, each R^{qq} where present is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{qq} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Most typically, each R^{qq} where present is hydrogen. In such an embodiment, Q³ may be selected from O, S or NH. Most typically, Q³ is NH.

As stated, G is —O—, —C(R^{g})₂—, or —NR^{gg}—. Typically, G is —O—, —CH₂—, or —NH—.

In one embodiment, G is —O— or —NR^{gg}—. Typically in such an embodiment, G is —O— or —NH—.

In another embodiment, G is —NR^{gg}—. Most typically, G is —NH—.

In one embodiment, each R^{g} where present is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton.

In a further embodiment, each R^{g} where present is independently selected from hydrogen or a halo, —OH, —NH₂, —SH, or a saturated or unsaturated C₁-C₁₂ hydrocarbyl group, wherein the C₁-C₁₂ hydrocarbyl group may be straight-chained or branched, or be or include one or more cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{g} is optionally substituted, typically it is substituted with one or more groups independently selected from halo, —CN, —OH, —NH₂, —N(O)(R^{qp})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

More typically, each R^{g} where present is independently selected from hydrogen or a halo, —OH, —NH₂, —CN, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated C₁-C₆ hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically still, each R^{g} where present is independently selected from hydrogen or a halo, —OH, —NH₂, —CN, —R^{gx}, —OR^{gx}, —NHR^{gx} or —N(R^{gx})₂ group, wherein each R^{gx} is independently selected from a C₁-C₄ alkyl, C₁-C₄ haloalkyl, C₃-C₄ cycloalkyl or C₃-C₄ halocycloalkyl group, or any two R^{gx} directly attached to the same nitrogen atom may together form a C₂-C₅ alkylene or C₂-C₅ haloalkylene group. Typically at least one R^{gx} in any —C(R^{gx})₂— group is selected from hydrogen or a halo, —CN or —R^{gx} group.

Yet more typically, a first R^{g} in any —C(R^{g})₂— group is independently selected from hydrogen or a fluoro, chloro, -Me or -Et group, and the second R^{g} in the —C(R^{g})₂— group is independently selected from hydrogen or a fluoro, chloro, —OH, —NH₂, -Me, -Et, —OMe, —OEt, —NHMe, —NHEt, —N(Me)₂, —N(Me)Et or —N(Et)₂ group, wherein any methyl (Me) or ethyl (Et) group may optionally be substituted with one or more fluoro and/or chloro groups.

In another embodiment, any two R^{g} may, together with the carbon atom to which they are attached, form a cyclic group, wherein the cyclic group may optionally be substituted. For example, any two R^{g}, together with the carbon atom to which they are attached, may form a saturated or unsaturated 3- to 12-membered cyclic group, wherein the 3- to 12-membered cyclic group may optionally be substituted. Where the 3- to 12-membered cyclic group is optionally substituted, typically it is substituted with one or more groups independently selected from halo, —CN, —OH, —NO₂, —NH₂, oxo (═O), ═NH, —R^{gy}, —OR^{gy}, —NHR^{gy}, —N(R^{gy})₂, —N(O)(R^{gy})₂, —N⁺(R^{gy})₃ or ═NR^{gy}, wherein each R^{gy} is independently selected from a C₁-C₄ alkyl, C₁-C₄ haloalkyl, C₃-C₄ cycloalkyl or C₃-C₄ halocycloalkyl group, or any two R^{gy} directly attached to the same nitrogen atom may together form a C₂-C₅ alkylene or C₂-C₅ haloalkylene group.

More typically, any two R^{g}, together with the carbon atom to which they are attached, may form a 3- to 7-membered saturated or unsaturated monocyclic group, wherein the monocyclic group may optionally be substituted with one or more groups independently selected from halo, —CN, —OH, —NO₂, —NH₂, oxo (═O), ═NH, -Me, -Et, —OMe, —OEt, —NH Me, —NHEt, —N(Me)₂, —N(Me)Et, —N(Et)₂, —N⁺(Me)₃, —N⁺(Me)₂Et, —N⁺(Et)₂Me or —N⁺(Et)₃, wherein any methyl (Me) or ethyl (Et) group may optionally be substituted with one or more halo groups.

More typically still, any two R^{g} may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. For example, in one embodiment any two R^{g} may, together with the carbon atom to which they are attached, form a cyclopropyl group, wherein the cyclopropyl group may optionally be substituted with one or more fluoro groups.

In one embodiment, each R^{g} where present is independently selected from hydrogen or a fluoro, chloro, -Me or -Et group, wherein any methyl (Me) or ethyl (Et) group may optionally be substituted with one or more fluoro and/or chloro groups, or any two R^{g} may, together with the carbon atom to which they are attached, form a 3- or 4-membered cycloalkyl group, or form an oxetanyl group, wherein the 3- or 4-membered cycloalkyl group or the oxetanyl group may optionally be substituted with one or more fluoro and/or chloro groups. Typically in such an embodiment, each R^{g} where present is independently selected from hydrogen or a fluoro or methyl group, wherein the methyl group may optionally be substituted with one or more fluoro groups, or any two R^{g} may, together with the carbon atom to which they are attached, form a cyclopropyl group, wherein the cyclopropyl group may optionally be substituted with one or more fluoro groups.

Yet more typically still, each R^{g} where present is independently selected from hydrogen or a fluoro or methyl group, wherein the methyl group may optionally be substituted with one or more fluoro groups. Most typically, each R^{g} where present is hydrogen.

As stated, each R^{gg} where present is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton.

In one embodiment, each R^{gg} where present is independently selected from hydrogen or a saturated or unsaturated C₁-C₁₂ hydrocarbyl group, wherein the C₁-C₁₂ hydrocarbyl group may be straight-chained or branched, or be or include one or more cyclic groups, wherein the C₁-C₁₂ hydrocarbyl group may optionally be substituted, and wherein the C₁-C₁₂ hydrocarbyl group may optionally include one, two or three heteroatoms N, O or S in its carbon skeleton. Where the hydrocarbyl group of R^{gg} is optionally substituted, typically it is substituted with one or more groups independently selected from halo, —CN, —OH, —NH₂, —N(O)(R^{qp})₂, —N⁺(R^{qp})₃, oxo (═O) and ═NH, wherein each R^{qp} is independently selected from a C₁-C₄ alkyl, C₁-C₄ haloalkyl, C₃-C₄ cycloalkyl or C₃-C₄ halocycloalkyl group, or any two R^{qp} directly attached to the same nitrogen atom may together form a C₂-C₅ alkylene or C₂-C₅ haloalkylene group.

More typically, each R^{gg} where present is independently selected from hydrogen or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally be substituted with one or more groups independently selected from halo, —CN, —OH, —NH₂, —N⁺(R^{qp})₃ and oxo (═O), wherein the saturated C₁-C₆ hydrocarbyl group may optionally include one or two heteroatoms N or O in its carbon skeleton, and wherein each R^{qp} is independently selected from a methyl or an ethyl group, wherein any methyl (Me) or ethyl (Et) group may optionally be substituted with one or more halo groups.

More typically still, each R^{gg} where present is independently selected from hydrogen or a C₁-C₄ alkyl or C₃-C₄ cycloalkyl group, wherein the C₁-C₄ alkyl or C₃-C₄ cycloalkyl group may optionally be substituted with one or more fluoro and/or chloro groups. For example, each R^{gg} where present may be independently selected from hydrogen or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, each R^{gg} where present is independently selected from hydrogen or a methyl group, wherein the methyl group may optionally be substituted with one or more fluoro groups. Most typically, each R^{gg} where present is hydrogen.

In one embodiment, the first aspect of the invention provides a compound of formula (Ia):

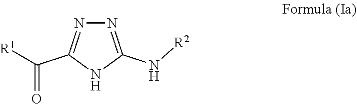

wherein R¹ and R² are as defined herein.

As stated, R¹ is hydrogen, —OH, —NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton. More typically, R¹ is —NH₂ or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton.

In one embodiment, R¹ is a saturated or unsaturated C₁-C₂₀ or C₁-C₁₅ or C₁-C₁₂ hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton. Typically in such an embodiment, the atom of R¹ that is directly attached to the carbon atom of the carbonyl group of formula (I) or (Ia) is an oxygen or a nitrogen atom.

In a further embodiment, R¹ is selected from R¹⁰—O—, (R¹⁰)NH— or (R¹⁰)₂N—, wherein each R¹⁰ is independently selected from a C₁-C₁₂ alkyl, C₂-C₁₂ alkenyl, C₂-C₁₂ alkynyl or R¹¹-L- group, wherein R¹¹ is a 3- to 12-membered cyclic group and L is a bond or a C₁-C₆ alkylene, C₂-C₆ alkenylene, or C₂-C₁₂ alkynylene group, or two R¹⁰ may, together with the nitrogen atom to which they are attached, form a 3- to 12-membered heterocyclic group, wherein any alkyl, alkenyl, alkynyl, alkylene, alkenylene or alkynylene group may optionally include one or more heteroatoms independently selected from oxygen and nitrogen in their carbon skeleton, and wherein any alkyl, alkenyl, alkynyl, alkylene, alkenylene, alkynylene, cyclic or heterocyclic group may optionally be substituted.

Typically in such an embodiment, each R¹⁰ is independently selected from a C₁-C₈ alkyl, C₂-C₅ alkenyl or R¹¹-L- group, wherein R¹¹ is a 3- to 7-membered monocyclic group or a 7- to 10-membered fused bicyclic group and L is a bond or a C₁-C₄ alkylene or C₂-C₄ alkenylene group, or two R¹⁰ may, together with the nitrogen atom to which they are attached, form a 3- to 7-membered monocyclic heterocyclic group or a 7- to 10-membered fused bicyclic heterocyclic group, wherein any alkyl, alkenyl, alkylene or alkenylene group may optionally include one or two heteroatoms independently selected from oxygen and nitrogen in their carbon skeleton, and wherein any alkyl, alkenyl, alkylene, alkenylene, monocyclic or fused bicyclic group may optionally be substituted.

Typically in the above embodiment, R¹ is R¹⁰—O—.

In a further embodiment, R¹ is R¹⁰—O—, wherein R¹⁰ is selected from a C₁-C₆ alkyl or R¹¹-L- group, wherein R¹¹ is a 3- to 7-membered monocyclic group or a 7- to 10-membered fused bicyclic group and L is a bond or a C₁-C₃ alkylene group, wherein any alkyl, alkylene, monocyclic or fused bicyclic group may optionally be substituted.

Where R¹ is a 3- to 7-membered monocyclic group, typically the monocyclic group is selected from a cycloalkyl, cycloalkenyl, non-aromatic heterocyclic, phenyl or heteroaryl group.

Where R¹¹ is a 7- to 10-membered fused bicyclic group, typically the bicyclic group is selected from a naphthyl, heteroaryl, cycloalkyl, cycloalkenyl, non-aromatic heterocyclic or partially aromatic bicyclic group.

Where an alkyl, alkenyl, alkynyl, alkylene, alkenylene or alkynylene group of R¹ or R¹⁰ is substituted, typically it is substituted with one or more substituents independently selected from halo, —CN, —OH, —NH₂, oxo (═O) and ═NH. More typically, where an alkyl, alkenyl, alkynyl, alkylene, alkenylene or alkynylene group of R¹ or R¹⁰ is substituted, it is substituted with one or more substituents independently selected from halo, —CN, —OH, —NH₂ and oxo (═O). Yet more typically, where an alkyl, alkenyl, alkynyl, alkylene, alkenylene or alkynylene group of R¹ or R¹⁰ is substituted, it is substituted with one or more substituents independently selected from fluoro and oxo (═O).

Typically, where an alkyl, alkenyl, alkynyl, alkylene, alkenylene or alkynylene group of R¹ or R¹⁰ is substituted, it is substituted with a maximum of three non-halo substituents.

Where a cyclic group of R¹, R¹⁰ or R¹¹, such as a 3- to 12-membered cyclic group, a 3- to 12-membered heterocyclic group, a 3- to 7-membered monocyclic group or a 7- to 10-membered fused bicyclic group, is substituted, typically it is substituted with one or more substituents independently selected from halo, —CN, —OH, —NO₂, —NH₂, oxo (═O), ═NH, —R¹⁰¹, —OR¹⁰¹, —NHR¹⁰¹, —N(R¹⁰¹)₂, —N(O)(R¹⁰¹)₂, —N⁺(R¹⁰¹)₃ or ═NR¹⁰¹, wherein each R¹⁰¹ is independently selected from a C₁-C₄ alkyl, C₁-C₄ haloalkyl, C₃-C₄ cycloalkyl or C₃-C₄ halocycloalkyl group, or any two R¹⁰¹ directly attached to the same nitrogen atom may together form a C₂-C₅ alkylene or C₂-C₅ haloalkylene group. More typically, where a cyclic group of R¹, R¹⁰ or R¹¹ is substituted, it is substituted with one or more substituents independently selected from halo, —CN, —OH, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups.

Typically, where a cyclic group of R¹, R¹⁰ or R¹¹ is substituted, it is substituted with a maximum of three non-halo substituents.

In another embodiment:
R¹ is R¹⁰—O—, wherein R¹⁰ is selected from a C₁-C₆ alkyl, C₁-C₆ haloalkyl, C₃-C₆ cycloalkyl or C₃-C₆ halocycloalkyl group; or
R¹ is R¹¹—CH₂—O—, wherein R¹¹ is selected from a phenyl, halophenyl or 5- or 6-membered heteroaryl group, wherein the 5- or 6-membered heteroaryl group may optionally be halo-substituted.

More typically still:
R¹ is R¹⁰—O—, wherein R¹⁰ is selected from a methyl, ethyl or isopropyl group; or
R¹ is R¹¹—CH₂—O—, wherein R¹¹ is selected from a phenyl or 6-membered heteroaryl group.

In one aspect of any of the above embodiments, R¹ contains only atoms selected from the group consisting of carbon, hydrogen, nitrogen, oxygen and halogen atoms. Typically, R¹ contains only atoms selected from the group consisting of carbon, hydrogen, nitrogen, oxygen and fluorine atoms.

In another aspect of any of the above embodiments, R¹ contains from 1 to 30 atoms other than hydrogen or halogen. Typically, R¹ contains from 1 to 20 atoms other than hydrogen or halogen. More typically, R¹ contains from 1 to 15 atoms other than hydrogen or halogen. More typically still, R¹ contains from 1 to 10 atoms other than hydrogen or halogen.

As stated, R² is a cyclic group substituted at the α-position, wherein R² may optionally be further substituted. For the avoidance of doubt, it is noted that it is a ring atom of the cyclic group of R² that is directly attached to the group G, not any substituent.

In one embodiment of the first aspect of the invention, R² is an aryl or a heteroaryl group, wherein the aryl or the heteroaryl group is substituted at the α-position, and wherein R² may optionally be further substituted. Typically, R² is a phenyl or a 5- or 6-membered heteroaryl group, wherein the phenyl or the heteroaryl group is substituted at the α-position, and wherein R² may optionally be further substituted. Typically, R² is an aryl or a heteroaryl group, wherein the aryl or the heteroaryl group is substituted at the α and α′ positions, and wherein R² may optionally be further substituted. Typically, R² is a phenyl or a 5- or 6-membered heteroaryl group, wherein the phenyl or the heteroaryl group is substituted at the α and α′ positions, and wherein R² may optionally be further substituted. For example, R² may be a phenyl group substituted at the 2- and 6-positions or a phenyl group substituted at the 2-, 4- and 6-positions.

In one embodiment, the parent phenyl or 5- or 6-membered heteroaryl group of R² may be selected from phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl. Typically, the parent phenyl or 5- or 6-membered heteroaryl group of R² may be selected from phenyl, pyridinyl, pyridazinyl, pyrimidinyl, pyrrolyl, pyrazolyl, imidazolyl or triazolyl. Typically, the parent phenyl or 5- or 6-membered heteroaryl group of R² may be selected from phenyl, pyridinyl, pyridazinyl, pyrimidinyl or pyrazolyl. More typically, the parent phenyl or 5- or 6-membered heteroaryl group of R² may be selected from phenyl or pyrazolyl.

As used herein, the nomenclature α, β, α′, β′ refers to the position of the atoms of a cyclic group, such as —R², relative to the point of attachment of the cyclic group to the remainder of the molecule. For example, where —R² is a 1,2,3,5,6,7-hexahydro-s-indacen-4-yl moiety, the α, β, α′ and β′ positions are as follows:

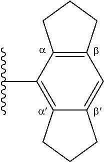 [SMILES: *c1c2c(cc3c1CCC3)CCC2]

For the avoidance of doubt, where it is stated that a cyclic group, such as an aryl or a heteroaryl group, is substituted at the α and/or α′ positions, it is to be understood that one or more hydrogen atoms at the α and/or α′ positions respectively are replaced by one or more substituents, such as any optional substituent as defined above. Unless stated otherwise, the term “substituted” does not include the replacement of one or more ring carbon atoms by one or more ring heteroatoms.

In another embodiment, R² is a cyclic group substituted at the α and α′ positions, wherein R² may optionally be further substituted. For example, R² may be a cycloalkyl, cycloalkenyl or non-aromatic heterocyclic group substituted at the α and α′ positions.

In any of the above embodiments, typical substituents at the α and/or α′ positions of the parent cyclic group of R² comprise a carbon atom. For example, typical substituents at the α and/or α′ positions may be independently selected from —R⁴, —OR⁴ and —COR⁴ groups, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. More typically, the substituents at the α and/or α′ positions are independently selected from alkyl and cycloalkyl groups, such as C₃-C₆ branched alkyl and C₃-C₆ cycloalkyl groups, e.g. isopropyl, cyclopropyl, cyclohexyl or t-butyl groups, wherein the alkyl and cycloalkyl groups are optionally further substituted with one or more fluoro and/or chloro groups.

In one aspect of any of the above embodiments, at least one substituent at the α and/or α′ positions comprises a carbon atom. Typically, each substituent at the α and/or α′ positions comprises a carbon atom. More typically, R² is substituted at the α and α′ positions and both substituents at the α and α′ positions comprise a carbon atom.

In a further aspect of any of the above embodiments, at least one substituent at the α and/or α′ positions comprises a sp² or sp³ hybridised carbon atom. Typically, each substituent at the α and/or α′ positions comprises a sp² or sp³ hybridised carbon atom. More typically, R² is substituted at the α and α′ positions and both substituents at the α and α′ positions comprise a sp² or sp³ hybridised carbon atom.

Typically, at least one substituent at the α and/or α′ positions comprises a sp³ hybridised carbon atom.

Other typical substituents at the α and/or α′ positions of the parent cyclic group of R² may include cycloalkyl, cycloalkenyl, non-aromatic heterocyclic, aryl or heteroaryl rings which are fused to the parent cyclic group across the α,β and/or α′,β′ positions respectively. Such fused cyclic groups are described in greater detail below.

In one embodiment, R² is a fused aryl or a fused heteroaryl group, wherein the aryl or heteroaryl group is fused to one or more cycloalkyl, cycloalkenyl, non-aromatic heterocyclic, aryl or heteroaryl rings, wherein R² may optionally be further substituted.

Typically, a cycloalkyl, cycloalkenyl, non-aromatic heterocyclic, aryl or heteroaryl ring is fused to the aryl or heteroaryl group across the α,β positions. Typically, the aryl or heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

More typically, R² is a fused phenyl or a fused 5- or 6-membered heteroaryl group, wherein the phenyl or the 5- or 6-membered heteroaryl group is fused to one or more cycloalkyl, cycloalkenyl, non-aromatic heterocyclic, aryl or heteroaryl rings, wherein R² may optionally be further substituted. Typically, a cycloalkyl, cycloalkenyl, non-aromatic heterocyclic, aryl or heteroaryl ring is fused to the phenyl or the 5- or 6-membered heteroaryl group across the α,β positions so as to form a 4- to 6-membered fused ring structure. Typically, the phenyl or the 5- or 6-membered heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

In another embodiment, R² is a fused aryl or a fused heteroaryl group, wherein the aryl or heteroaryl group is fused to two or more independently selected cycloalkyl, cycloalkenyl, non-aromatic heterocyclic, aryl or heteroaryl rings, wherein R² may optionally be further substituted. Typically, the two or more cycloalkyl, cycloalkenyl, non-aromatic heterocyclic, aryl or heteroaryl rings are each ortho-fused to the aryl or heteroaryl group, i.e. each fused cycloalkyl, cycloalkenyl, non-aromatic heterocyclic, aryl or heteroaryl ring has only two atoms and one bond in common with the aryl or heteroaryl group. Typically in such an embodiment, R² is tricyclic.

In yet another embodiment, R² is a fused aryl or a fused heteroaryl group, wherein a first cycloalkyl, cycloalkenyl, non-aromatic heterocyclic, aryl or heteroaryl ring is fused to the aryl or heteroaryl group across the α,β positions and a second cycloalkyl, cycloalkenyl, non-aromatic heterocyclic, aryl or heteroaryl ring is fused to the aryl or heteroaryl group across the α′,β′ positions, wherein R² may optionally be further substituted. Typically in such an embodiment, R² is tricyclic.

More typically, R² is a fused phenyl or a fused 5- or 6-membered heteroaryl group, wherein a first cycloalkyl, cycloalkenyl, non-aromatic heterocyclic, aryl or heteroaryl ring is fused to the phenyl or the 5- or 6-membered heteroaryl group across the α,β positions so as to form a first 4- to 6-membered fused ring structure, and a second cycloalkyl, cycloalkenyl, non-aromatic heterocyclic, aryl or heteroaryl ring is fused to the phenyl or the 5- or 6-membered heteroaryl group across the α′,β′ positions so as to form a second 4- to 6-membered fused ring structure, wherein R² may optionally be further substituted. Typically in such an embodiment, R² is tricyclic.

In one embodiment, —R² has a formula selected from:

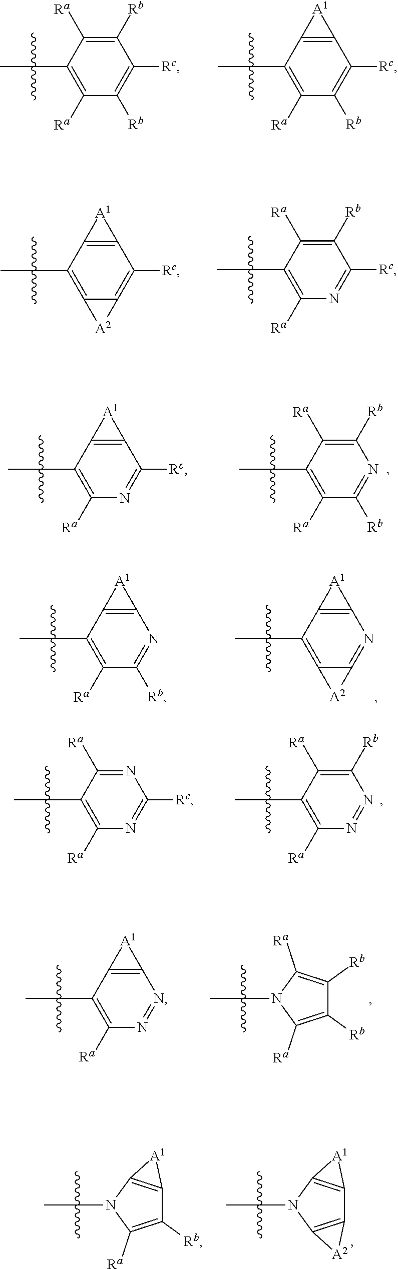

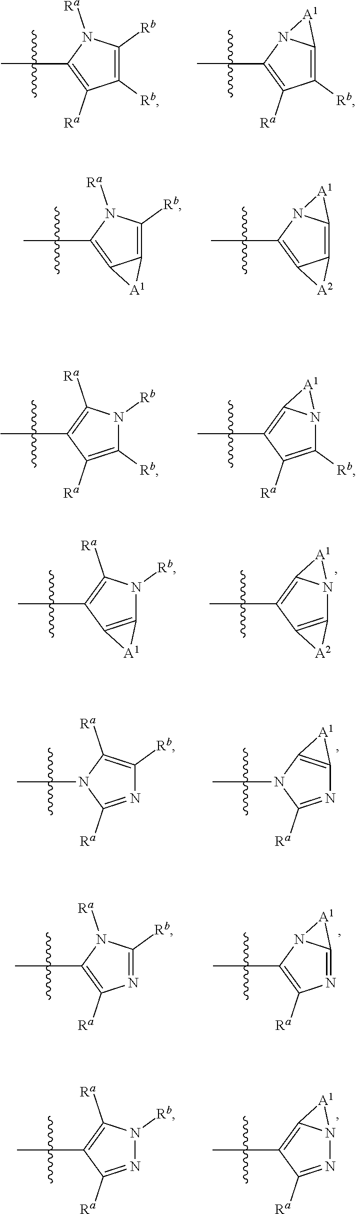

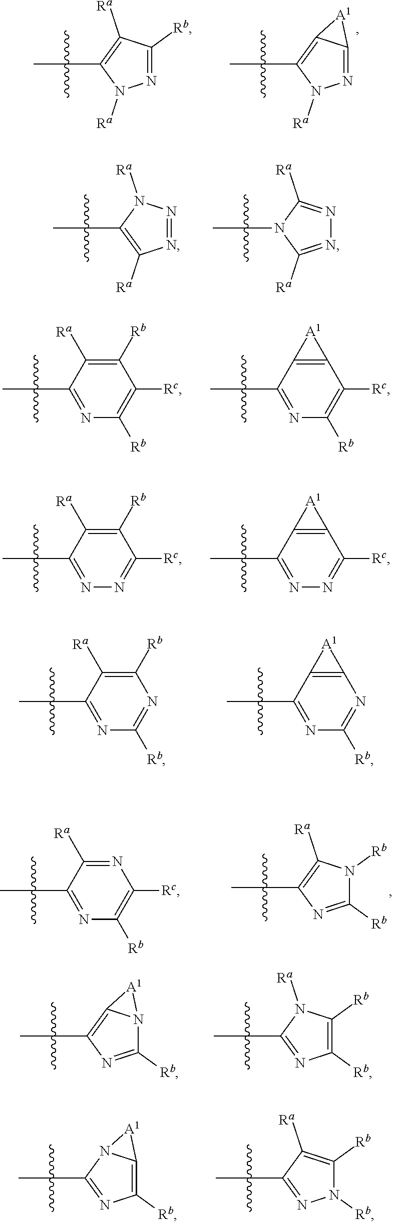

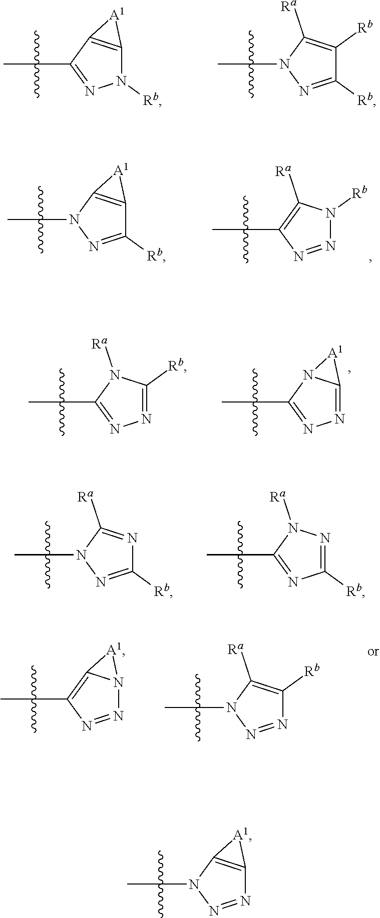

wherein:
A¹ and A² are each independently selected from an optionally substituted alkylene or alkenylene group, wherein one or more carbon atoms in the backbone of the alkylene or alkenylene group may optionally be replaced by one or more heteroatoms N, O or S;
each R^{a} is independently selected from hydrogen, halo, —R^{aa}, —OR^{aa} or —COR^{aa}, provided that at least one R^{a} is —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{c} is independently selected from hydrogen, halo, —OH, —NO₂, —CN, —R^{c}, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc};
each R^{aa} is independently selected from a C₁-C₆ alkyl, C₂-C₆ alkenyl, C₂-C₆ alkynyl or a 3- to 7-membered cyclic group, wherein each R^{aa} is optionally substituted; and
each R^{cc} is independently selected from a C₁-C₆ alkyl, C₂-C₆ alkenyl, C₂-C₆ alkynyl or a 3- to 7-membered cyclic group, or any two R^{cc} attached to the same nitrogen atom may, together with the nitrogen atom to which they are attached, form a 3- to 7-membered heterocyclic group, wherein each R^{cc} is optionally substituted.

Typically in the above embodiment, —R² has a formula selected from:

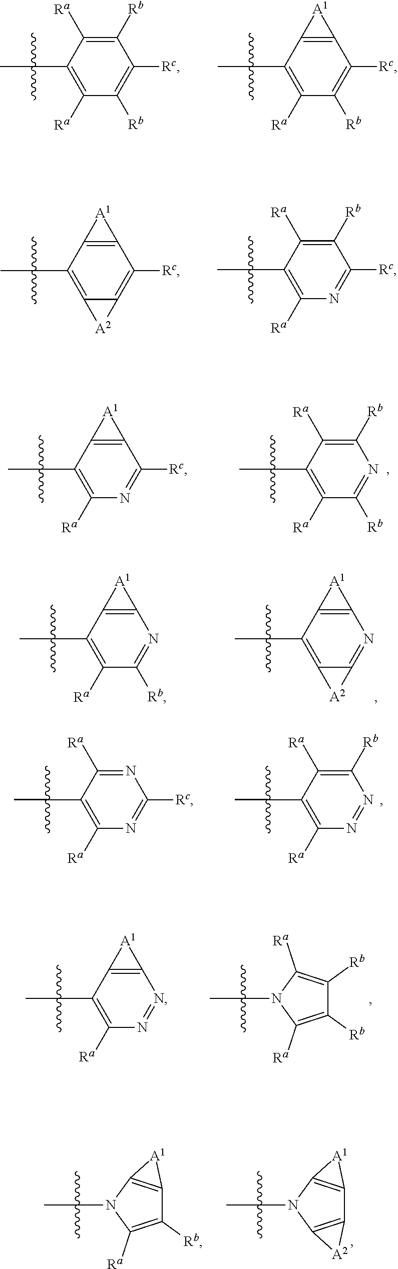

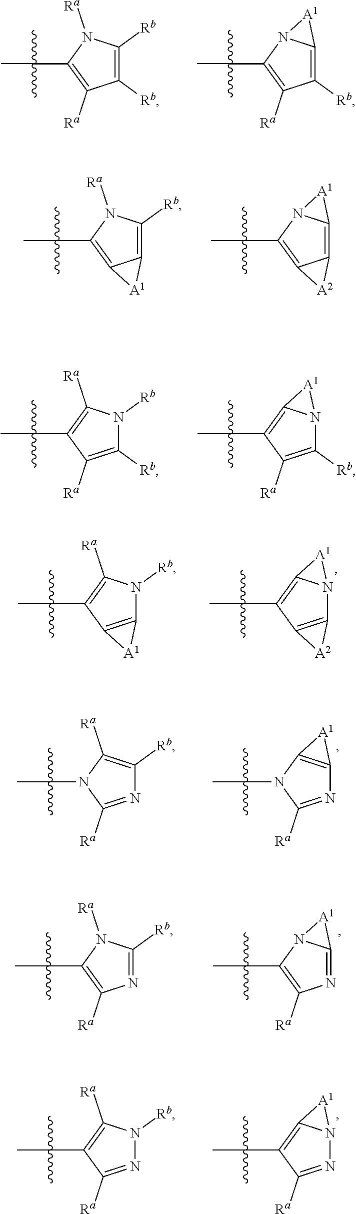

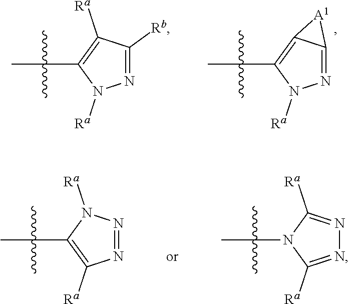

wherein each R^{a} is independently selected from —R^{aa}, —OR^{aa} or —COR^{aa}, and R^{aa}, R^{b}, R^{c}, A¹ and A² are as defined above.

More typically in the above embodiment, —R² has a formula selected from:

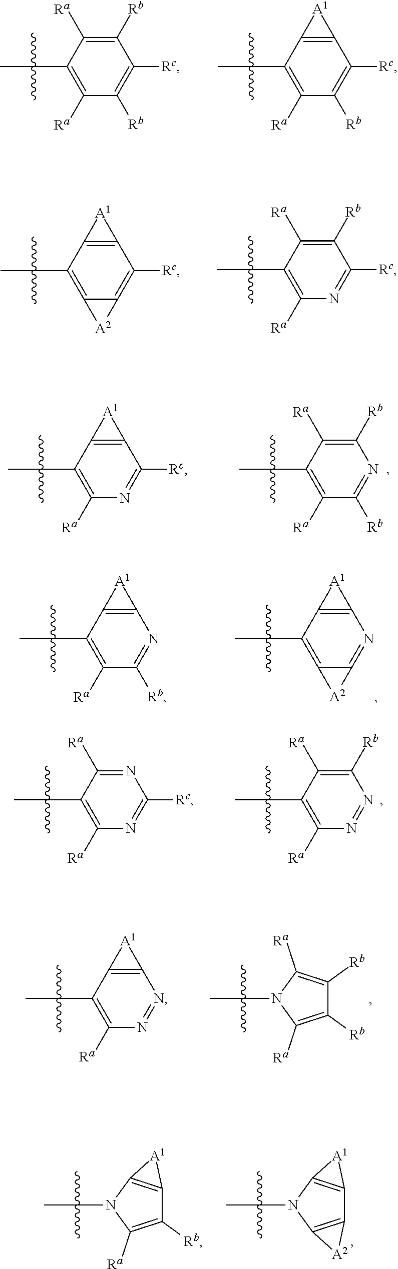

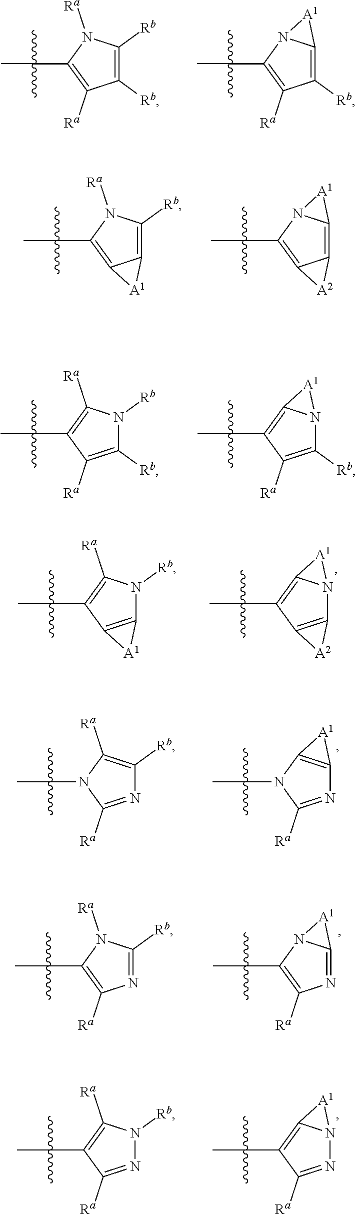

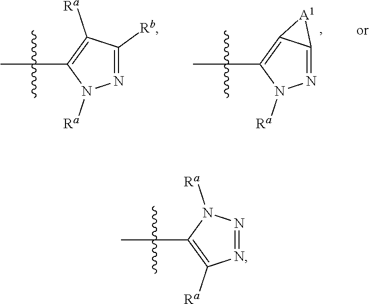

wherein each R^{a} is independently selected from —R^{aa}, —OR^{aa} or —COR^{aa}, and R^{aa}, R^{b}, R^{c}, A¹ and A² are as defined above.

Typically, R² is not connected to G via an oxygen-nitrogen or a nitrogen-nitrogen bond. For example, where G is —O— or —NR^{gg}—, —R² may have a formula selected from:

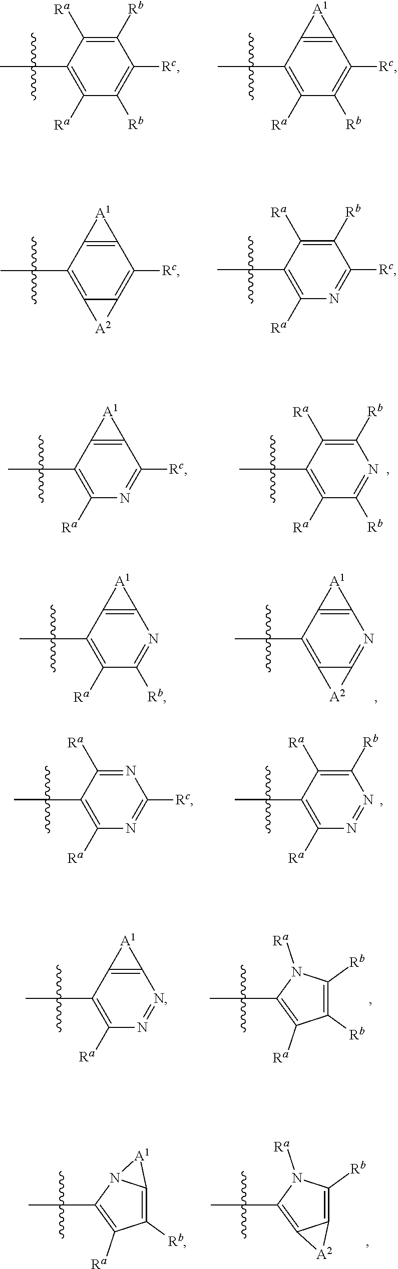

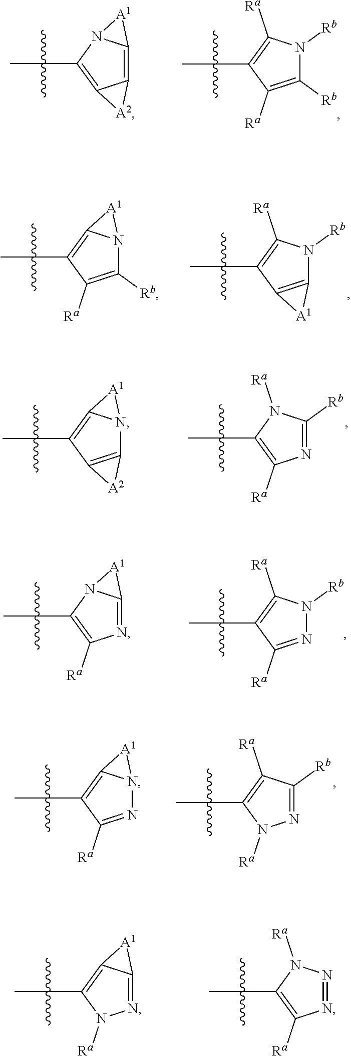

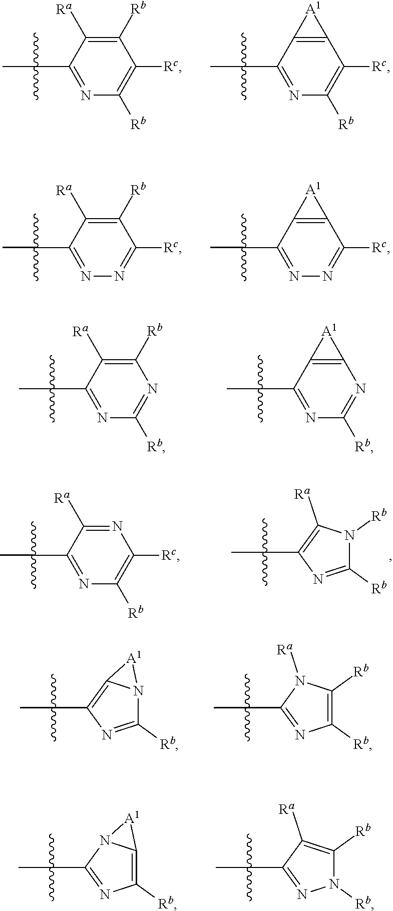

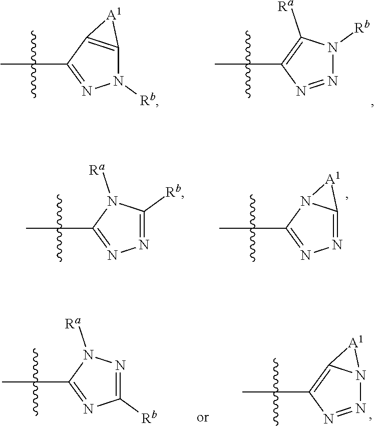

wherein R^{a}, R^{b}, R^{c}, A¹ and A² are as defined above.

More typically, where G is —O— or —NR^{gg}—, —R² has a formula selected from:

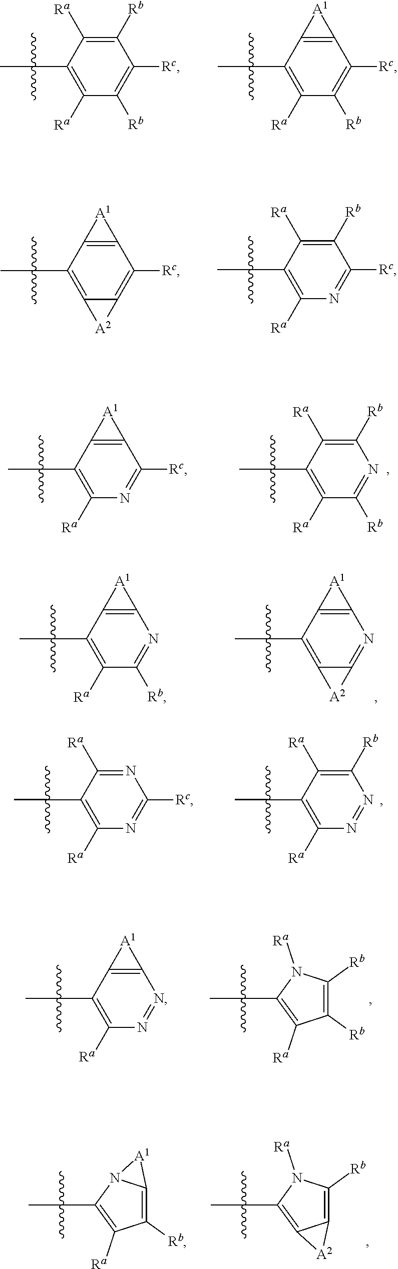

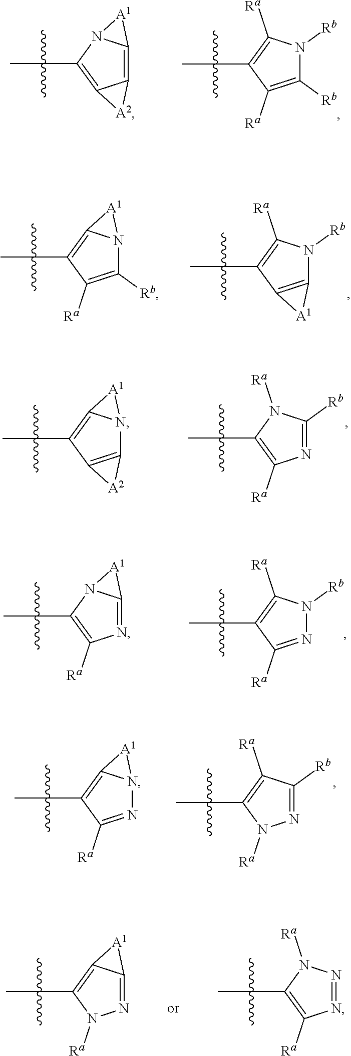

wherein each R^{a} is independently selected from —R^{aa}, —OR^{aa} or —COR^{aa}, and R^{aa}, R^{b}, R^{c}, A¹ and A² are as defined above.

More typically in any embodiment, —R² has a formula selected from:

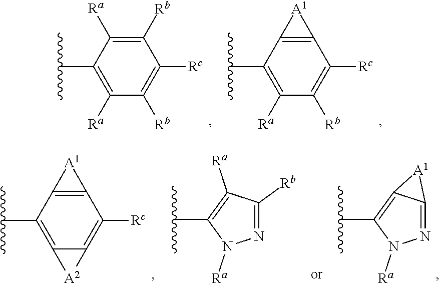

wherein R^{a}, R^{b}, R^{c}, A¹ and A² are as defined above.

More typically still, —R² has a formula selected from:

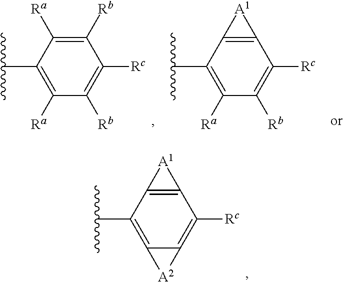

wherein each R^{a} is independently selected from —R^{aa}, —OR^{aa} or —COR^{aa}, and R^{aa}, R^{b}, R^{c}, A¹ and A² are as defined above.

Typically in any of the above embodiments, any ring containing A¹ or A² is a 5- or 6-membered ring. Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl). More typically, A¹ and A² are unsubstituted or substituted with one or more fluoro and/or chloro groups. Where R² contains both A¹ and A² groups, A¹ and A² may be the same or different. Typically, A¹ and A² are the same.

Where R^{aa} is a substituted C₁-C₆ alkyl, C₂-C₆ alkenyl or C₂-C₆ alkynyl group, typically the C₁-C₆ alkyl, C₂-C₆ alkenyl or C₂-C₆ alkynyl group is substituted with one or more (e.g. one or two) substituents independently selected from halo, —OH, —CN, —NO₂, —O(C₁-C₄ alkyl) or —O(C₁-C₄ haloalkyl).

Where R^{aa} is a substituted 3- to 7-membered cyclic group, typically the 3- to 7-membered cyclic group is substituted with one or more (e.g. one or two) substituents independently selected from halo, —OH, —NH₂, —CN, —NO₂, —B¹, —CH₂B¹, —OB¹, —OCH₂B¹, —NHB¹, —N(B¹)₂, —CONH₂, —CONHB¹, —CON(B¹)₂, —NHCOB¹, —NB¹COB¹, or —B¹¹—;
wherein each B¹ is independently selected from a C₁-C₆ alkyl, C₂-C₆ alkenyl, C₂-C₆ alkynyl, C₃-C₁₀ cycloalkyl, C₅-C₁₀ cycloalkenyl, C₆-C₁₀ aryl, or a 4- to 10-membered heterocyclic group containing one or two ring heteroatoms N and/or O, or two B¹ together with the nitrogen atom to which they are attached may form a 4- to 10-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂;
wherein each B¹¹ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B¹², —OB¹², —NHB¹² or —N(B¹²)₂; and
wherein each B¹² is independently selected from a C₁-C₃ alkyl or C₁-C₃ haloalkyl group. Typically, any divalent group —B¹¹— forms a 4- to 6-membered fused ring.

Typically in such an embodiment, each B¹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B¹ together with the nitrogen atom to which they are attached may form a 4- to 6-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂, and wherein B¹² is as defined above.

In one embodiment, each R^{aa} is independently selected from a C₁-C₄ alkyl or a 3- to 6-membered cyclic group, wherein each C₁-C₄ alkyl group is optionally substituted with one or more halo substituents and/or one or two substituents independently selected from —OH, —CN, —O(C₁-C₄ alkyl) or —O(C₁-C₄ haloalkyl), and wherein each 3- to 6-membered cyclic group is optionally substituted with one or more halo substituents and/or one or two substituents independently selected from halo, —OH, —CN, —B¹, —CH₂B¹, —OB¹ or —OCH₂B¹;
wherein each B¹ is independently selected from a C₁-C₄ alkyl, C₃-C₆ cycloalkyl or phenyl group, or a 4- to 6-membered heterocyclic group containing one or two ring heteroatoms N and/or O, and wherein any B¹ may optionally be halo-substituted and/or substituted with one or two substituents independently selected from —OH, —B¹² or —OB¹²; and
each B¹² is independently selected from a C₁-C₃ alkyl or C₁-C₃ haloalkyl group.

In one embodiment, each R^{a} is independently selected from hydrogen, halo or —R^{aa}, provided that at least one R^{a} is —R^{aa}. Typically, each R^{a} is —R^{aa}. In one embodiment, each R^{a} is independently selected from a C₁-C₆ alkyl (in particular C₃-C₆ branched alkyl) or C₃-C₆ cycloalkyl group, wherein each R^{a} is optionally further substituted with one or more halo groups. More typically, each R^{a} is independently selected from a C₁-C₄ alkyl, C₁-C₄ haloalkyl, C₃-C₄ cycloalkyl or C₃-C₄ halocycloalkyl group. Where a group R^{a} is present at both the α- and α′-positions, each R^{a} may be the same or different. Typically, each R^{a} is the same.

In one embodiment, each R^{b} is independently selected from hydrogen, halo, methyl or fluoromethyl. Typically, each R^{b} is independently selected from hydrogen or halo. More typically, each R^{b} is hydrogen.

In one embodiment, R^{c} is selected from hydrogen, halo, —OH, —NO₂, —CN, —R^{cc}, —R^{cx}, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc}, wherein each —R^{cc} is independently selected from C₁-C₄ alkyl, C₁-C₄ haloalkyl, C₃-C₄ cycloalkyl or C₃-C₄ halocycloalkyl, or any two R^{c} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{x} is selected from a 3- to 7-membered cyclic group, wherein the 3- to 7-membered cyclic group is optionally halo substituted. Typically, R^{c} is selected from hydrogen, halo, —CN, —R^{cc}, —R^{cc}, —OR^{cc}, —COR^{cc}, —C(═NOH)R^{cc} or —C(═NOR^{cc})R^{cc}, wherein each —R^{cc} is independently selected from C₁-C₃ alkyl, C₁-C₃ fluoroalkyl, cyclopropyl or fluorocyclopropyl, and wherein —R^{cx} is selected from a phenyl, halophenyl or a 5- or 6-membered heteroaryl group, wherein the 5- or 6-membered heteroaryl group is optionally halo substituted. More typically, R^{c} is independently selected from hydrogen, —CN or halo.

In one embodiment, —R² has a formula selected from:

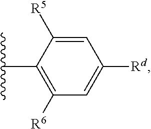

wherein R⁵ and R⁶ are independently selected from C₁-C₄ alkyl, C₁-C₄ haloalkyl, C₃-C₄ cycloalkyl and C₃-C₄ halocycloalkyl, and R^{d} is hydrogen, halo, —OH, —NO₂, —CN, —R^{dd}, —R^{dx}, —OR^{dd}, —COR^{dd}, —COOR^{dd}, —CONH₂, —CONHR^{dd}, —CON(R^{dd})₂, —C(═NH)R^{dd}, —C(═NH)NH₂, —C(═NH)NHR^{dd}, —C(═NH)N(R^{dd})₂, —C(═NR^{dd})R^{dd}, —C(═NR^{dd})NHR^{dd}, —C(═NR^{dd})N(R^{dd})₂, —C(═NOH)R^{dd} or —C(═NOR^{dd})R^{dd}, wherein each —R^{dd} is independently selected from C₁-C₄ alkyl, C₁-C₄ haloalkyl, C₃-C₄ cycloalkyl and C₃-C₄ halocycloalkyl, or any two R^{dd} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{dx} is selected from a 3- to 7-membered cyclic group, wherein the 3- to 7-membered cyclic group is optionally halo substituted. Typically, R⁵ and R⁶ are independently selected from C₁-C₄ alkyl, and R^{d} is hydrogen, halo, —CN, —R^{dd}, —R^{dx}, —OR^{dd}, —COR^{dd}, —C(═NOH)R^{dd} or —C(═NOR^{dd})R^{dd}, wherein each —R^{dd} is independently selected from C₁-C₃ alkyl, C₁-C₃ fluoroalkyl, cyclopropyl or fluorocyclopropyl, and wherein —R^{dx} is selected from a phenyl, halophenyl or a 5- or 6-membered heteroaryl group, wherein the 5- or 6-membered heteroaryl group is optionally halo substituted. More typically, R⁵ and R⁶ are independently selected from C₁-C₄ alkyl, and R^{d} is hydrogen or a halo group.

Typically, —R² has a formula selected from:

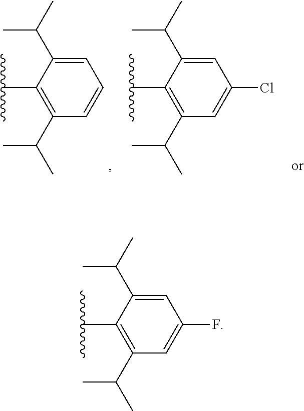

In one embodiment, —R² has a formula selected from:

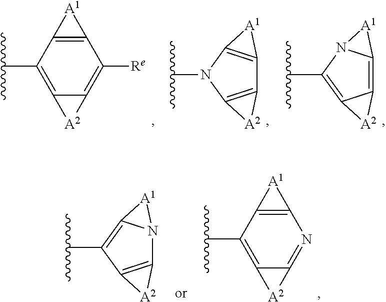

wherein A¹ and A² are each independently selected from an optionally substituted alkylene or alkenylene group, wherein one or more carbon atoms in the backbone of the alkylene or alkenylene group may optionally be replaced by one or more heteroatoms N, O or S, and wherein R^{e} is hydrogen or any optional substituent. R^{e} and any optional substituent attached to A¹ or A² may together with the atoms to which they are attached form a further fused cycloalkyl, cycloalkenyl, non-aromatic heterocyclic, aryl or heteroaryl ring which may itself be optionally substituted. Similarly, any optional substituent attached to A¹ and any optional substituent attached to A² may also together with the atoms to which they are attached form a further fused cycloalkyl, cycloalkenyl, non-aromatic heterocyclic, aryl or heteroaryl ring which may itself be optionally substituted.

In one embodiment, R^{e} is hydrogen, halo, —OH, —NO₂, —CN, —R^{ee}, —R^{ex}, —OR^{ee}, —COR^{ee}, —COOR^{ee}, —CONH₂, —CONHR^{ee}, —CON(R^{ee})₂, —C(═NH)R^{ee}, —C(═NH)NH₂, —C(═NH)NHR^{ee}, —C(═NH)N(R^{ee})₂, —C(═NR^{ee})R^{ee}, —C(═NR^{ee})NHR^{ee}, —C(═NR^{ee})N(R^{ee})₂, —C(═NOH)R^{ee} or —C(═NOR^{ee})R^{ee}, wherein each —R^{ee} is independently selected from C₁-C₄ alkyl, C₁-C₄ haloalkyl, C₃-C₄ cycloalkyl and C₃-C₄ halocycloalkyl, or any two R^{ee} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{ex} is selected from a 3- to 7-membered cyclic group, wherein the 3- to 7-membered cyclic group is optionally halo substituted. Typically, R^{e} is hydrogen, halo, —CN, —R^{ee}, —R^{ex}, —OR^{ee}, —COR^{ee}, —C(═NOH)R^{ee} or —C(═NOR^{ee})R^{ee}, wherein each —R^{ee} is independently selected from C₁-C₃ alkyl, C₁-C₃ fluoroalkyl, cyclopropyl or fluorocyclopropyl, and wherein —R^{ex} is selected from a phenyl, halophenyl or a 5- or 6-membered heteroaryl group, wherein the 5- or 6-membered heteroaryl group is optionally halo substituted.

In another embodiment, R^{e} is hydrogen or a halo, hydroxyl, —CN, —NO₂, —R^{ee} or —OR^{ee} group, wherein R^{ee} is a C₁-C₄ alkyl group which may optionally be halo-substituted. More typically, R^{e} is hydrogen or halo.

Typically, any ring containing A¹ or A² is a 5- or 6-membered ring. Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more halo, hydroxyl, —CN, —NO₂, —B³ or —OB³ groups, wherein B³ is a C₁-C₄ alkyl group which may optionally be halo-substituted. More typically, A¹ and A² are unsubstituted or substituted with one or more fluoro and/or chloro groups. Where R² contains both A¹ and A² groups, A¹ and A² may be the same or different. Typically, A¹ and A² are the same.

In a further embodiment, —R² has a formula selected from:

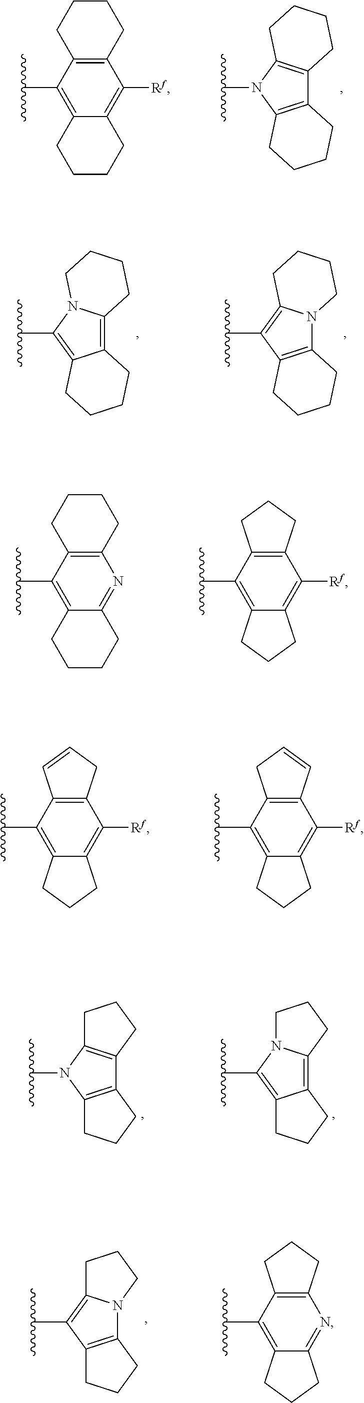

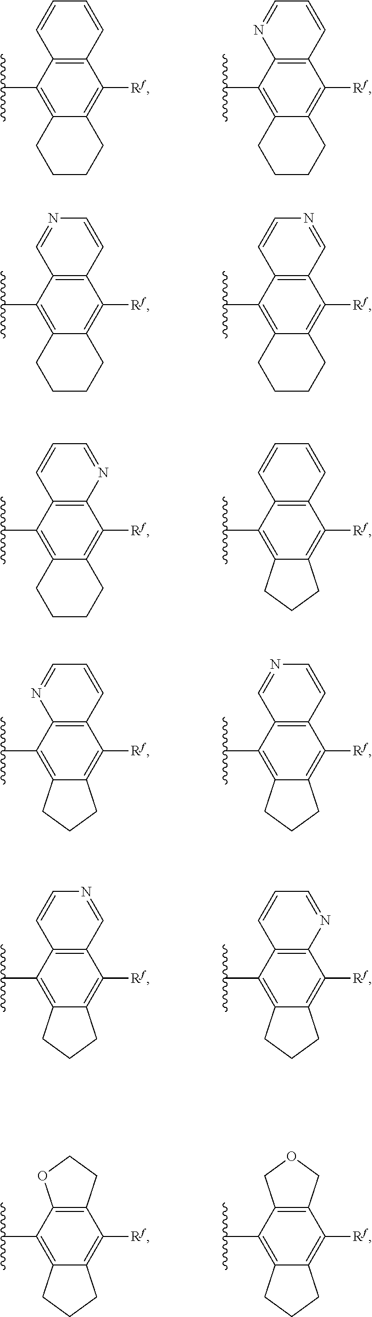

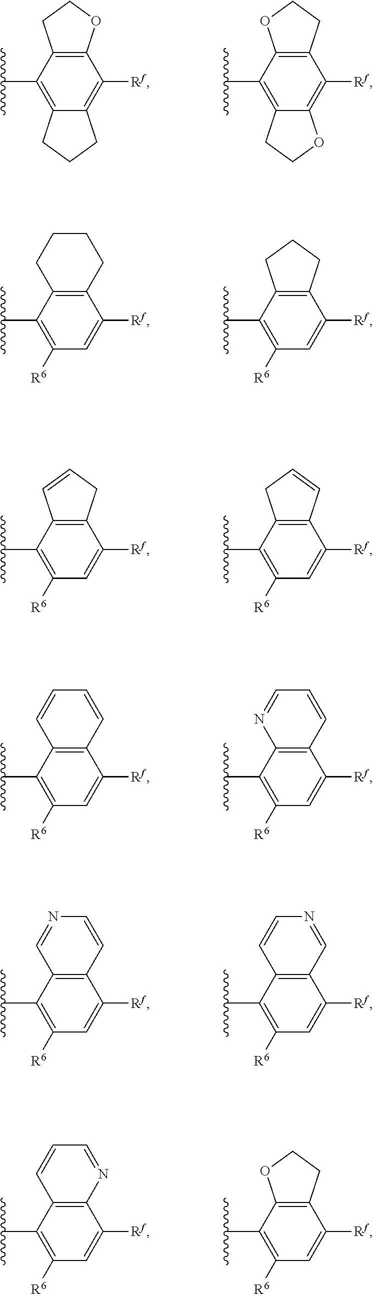

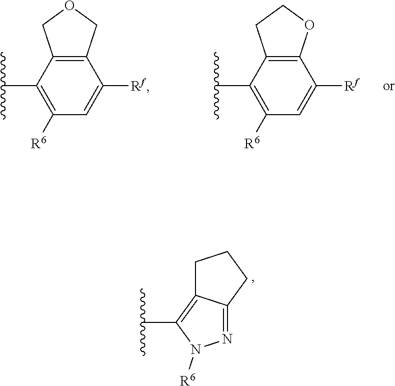

wherein R⁶ is C₁-C₄ alkyl, C₁-C₄ haloalkyl, C₃-C₄ cycloalkyl or C₃-C₄ halocycloalkyl, and R^{f} is hydrogen, halo, —OH, —NO₂, —CN, —R^{ff}, —R^{fx}, —OR^{ff}, —COR^{ff}, —COOR^{ff}, —CONH₂, —CONHR^{ff}, —CON(R^{ff})₂, —C(═NH)R^{ff}, —C(═NH)NH₂, —C(═NH)NHR^{ff}, —C(═NH)N(R^{ff})₂, —C(═NR^{ff})R^{ff}, —C(═NR^{ff})NHR^{ff}, —C(═NR^{ff})N(R^{ff})₂, —C(═NOH)R^{ff} or —C(═NOR^{ff})R^{ff}, wherein each —R^{ff} is independently selected from C₁-C₄ alkyl, C₁-C₄ haloalkyl, C₃-C₄ cycloalkyl and C₃-C₄ halocycloalkyl, or any two R^{ff} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{fx} is selected from a 3- to 7-membered cyclic group, wherein the 3- to 7-membered cyclic group is optionally halo substituted. Typically, R⁶ is C₁-C₄ alkyl, and R^{f} is hydrogen, halo, —CN, —R^{ff}, —R^{fx}, —OR^{ff}, —COR^{ff}, —C(═NOH)R^{ff} or —C(═NOR^{ff})R^{ff}, wherein each —R^{ff} is independently selected from C₁-C₃ alkyl, C₁-C₃ fluoroalkyl, cyclopropyl or fluorocyclopropyl, and wherein —R^{fx} is selected from a phenyl, halophenyl or a 5- or 6-membered heteroaryl group, wherein the 5- or 6-membered heteroaryl group is optionally halo substituted.

More typically, R⁶ is C₁-C₄ alkyl, and R^{f} is hydrogen or halo.

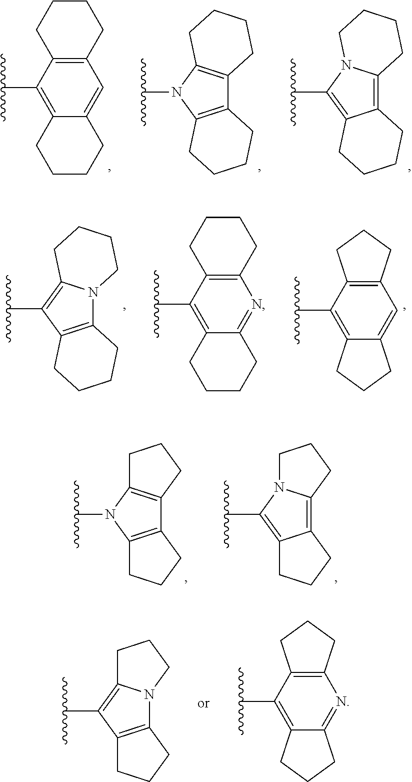

Typically, —R² has the formula:

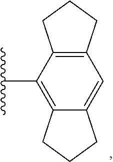 [SMILES: *c1c2c(cc3c1CCC3)CCC2]

Yet other typical substituents at the α-position of the parent cyclic group of R² may include monovalent heterocyclic groups and monovalent aromatic groups, wherein a ring atom of the heterocyclic or aromatic group is directly attached via a single bond to the α-ring atom of the parent cyclic group, wherein the heterocyclic or aromatic group may optionally be substituted, and wherein the parent cyclic group may optionally be further substituted. Such R² groups are described in greater detail below.

In one embodiment, the α-substituted parent cyclic group of R² is a 5- or 6-membered cyclic group, wherein the cyclic group may optionally be further substituted. In one embodiment, the α-substituted parent cyclic group of R² is an aryl or a heteroaryl group, all of which may optionally be further substituted. In one embodiment, the α-substituted parent cyclic group of R² is a phenyl or a 5- or 6-membered heteroaryl group, all of which may optionally be further substituted. In one embodiment, the α-substituted parent cyclic group of R² is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, all of which may optionally be further substituted. In one embodiment, the α-substituted parent cyclic group of R² is a phenyl or pyrazolyl group, both of which may optionally be further substituted. In a further embodiment, the α-substituted parent cyclic group of R² is a phenyl group, which may optionally be further substituted.

In one embodiment, the α-substituted parent cyclic group of R² is substituted at the α and α′ positions, and may optionally be further substituted. For example, the α-substituted parent cyclic group of R² may be a phenyl group substituted at the 2- and 6-positions or a phenyl group substituted at the 2-, 4- and 6-positions.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, the monovalent heterocyclic or aromatic group at the α-position is a phenyl or a 5- or 6-membered heterocyclic group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, thianyl, piperazinyl, 1,4-dioxanyl, morpholinyl or thiomorpholinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, piperidinyl or tetrahydropyranyl group, all of which may optionally be substituted. In one embodiment, the monovalent heterocyclic or aromatic group at the α-position is a phenyl, pyridinyl, pyridazinyl, pyrimidinyl, pyrazolyl, imidazolyl, isoxazolyl, thiazolyl, tetrahydropyranyl or 1-methyl-2-oxo-1,2-dihydropyridinyl group, all of which may optionally be substituted. In one embodiment, the monovalent heterocyclic or aromatic group at the α-position is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group at the α-position is a phenyl, pyridinyl, pyrimidinyl or pyrazolyl group, all of which may optionally be substituted. In one embodiment, the monovalent heterocyclic or aromatic group at the α-position is an unsubstituted phenyl, pyridinyl, pyrimidinyl or pyrazolyl group. In one embodiment, the monovalent heterocyclic group at the α-position is a pyridin-2-yl, pyridin-3-yl or pyridin-4-yl group, all of which may optionally be substituted. In one embodiment, the monovalent heterocyclic group at the α-position is an unsubstituted pyridin-3-yl group or an optionally substituted pyridin-4-yl group.

For any of these monovalent heterocyclic or aromatic groups at the α-position mentioned in the immediately preceding paragraph, the monovalent heterocyclic or aromatic group may optionally be substituted with one or two substituents independently selected from halo, —OH, —NH₂, —CN, —NO₂, —B⁴, —CH₂B⁴, —OB⁴, —OCH₂B⁴, —NHB⁴, —N(B⁴)₂, —CONH₂, —CONHB⁴, —CON(B⁴)₂, —NHCOB⁴, —NB⁴COB⁴, or —B⁴⁴—;
wherein each B⁴ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁴ together with the nitrogen atom to which they are attached may form a 4- to 6-membered heterocyclic group containing one or two ring heteroatoms N and/or O, wherein any B⁴ may optionally be halo-substituted and/or substituted with one or two substituents independently selected from —OH, —NH₂, —B⁴⁵, —OB⁴⁵, —NHB⁴⁵ or —N(B⁴⁵)₂;
wherein each B⁴⁴ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁴⁵, —OB⁴⁵, —NHB⁴⁵ or —N(B⁴⁵)₂; and
wherein each B⁴⁵ is independently selected from a C₁-C₃ alkyl or C₁-C₃ haloalkyl group.

Typically, any divalent group —B⁴⁴— forms a 4- to 6-membered fused ring.

In one embodiment, the monovalent heterocyclic or aromatic group at the α-position is a phenyl, pyridinyl, pyrimidinyl or pyrazolyl group, all of which may optionally be substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is a pyridin-2-yl, pyridin-3-yl or pyridin-4-yl group, all of which may optionally be substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is an unsubstituted pyridin-3-yl group or a pyridin-4-yl group optionally substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, such further substituents are in the α′ position of the α-substituted parent cyclic group of R². Such further substituents may be independently selected from halo, —R⁶, —OR⁶ or —COR⁶ groups, wherein each R⁶ is independently selected from a C₁-C₆ alkyl, C₂-C₆ alkenyl, C₂-C₆ alkynyl or C₂-C₆ cyclic group and wherein each R⁶ is optionally further substituted with one or more halo groups. Typically, such further substituents on the α-substituted parent cyclic group of R² are independently selected from halo, C₁-C₆ alkyl (in particular C₃-C₆ branched alkyl) or C₃-C₆ cycloalkyl groups, e.g. fluoro, chloro, isopropyl, cyclopropyl, cyclohexyl or t-butyl groups, wherein the alkyl and cycloalkyl groups are optionally further substituted with one or more fluoro and/or chloro groups.

In one embodiment, —R² has a formula selected from:

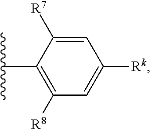

wherein R⁷ is C₁-C₄ alkyl, C₁-C₄ haloalkyl, C₃-C₆ cycloalkyl or C₃-C₆ halocycloalkyl, R⁸ is a 5- or 6-membered, optionally substituted heterocyclic or aromatic group, and R^{k} is hydrogen, halo, —OH, —NO₂, —CN, —R^{kk}, —R^{k}, —OR^{kk}, —COR^{kk}, —COOR^{kk}, —CONH₂, —CONHR^{kk}, —CON(R^{kk})₂, —C(═NH)R^{kk}, —C(═NH)NH₂, —C(═NH)NHR^{kk}, —C(═NH)N(R^{kk})₂, —C(═NR^{kk})R^{kk}, —C(═NR^{kk})NHR^{kk}, —C(═NR^{kk})N(R^{kk})₂, —C(═NOH)R^{kk} or —C(═NOR^{kk})R^{kk}, wherein each —R^{kk} is independently selected from C₁-C₄ alkyl, C₁-C₄ haloalkyl, C₃-C₄ cycloalkyl and C₃-C₄ halocycloalkyl, or any two R^{kk} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{k} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁵, —CH₂B⁵, —OB⁵, —OCH₂B⁵, —NHB⁵, —N(B⁵)₂, —CONH₂, —CONHB⁵, —CON(B⁵)₂, —NHCOB⁵, —NB⁵COB⁵, or —B⁵⁵—;
wherein each B⁵ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁵ together with the nitrogen atom to which they are attached may form a 4- to 6-membered heterocyclic group containing one or two ring heteroatoms N and/or O, wherein any B⁵ may optionally be halo-substituted and/or substituted with one or two substituents independently selected from —OH, —NH₂, —B⁵⁶, —OB⁵⁶, —NHB⁵⁶ or —N(B⁵⁶)₂;
wherein each B⁵⁵ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁵⁶, —OB⁵⁶, —NHB⁵⁶ or —N(B⁵⁶)₂; and
wherein each B⁵⁶ is independently selected from a C₁-C₃ alkyl or C₁-C₃ haloalkyl group.

Typically, any divalent group —B⁵⁵— forms a 4- to 6-membered fused ring. Typically, R⁷ is C₁-C₄ alkyl, R⁸ is a 5- or 6-membered, optionally substituted heterocyclic or aromatic group, and R^{k} is hydrogen, halo, —CN, —R^{kk}, —R^{kx}, —OR^{kk}, —COR^{kk}, —C(═NOH)R^{kk} or —C(═NOR^{kk})R^{kk}, wherein each —R^{kk} is independently selected from C₁-C₃ alkyl, C₁-C₃ fluoroalkyl, cyclopropyl or fluorocyclopropyl, and wherein —R^{kx} is selected from a phenyl, halophenyl or a 5- or 6-membered heteroaryl group, wherein the 5- or 6-membered heteroaryl group is optionally halo substituted. More typically, R⁷ is C₁-C₄ alkyl, R⁸ is a 5- or 6-membered, optionally substituted heterocyclic or aromatic group, and R^{k} is hydrogen or halo. In one embodiment, the optional substituents on the heterocyclic or aromatic group of R⁸ are independently selected from halo, —OH, —NH₂, —CN, —NO₂, —B⁵, —OB⁵, —NHB⁵ or —N(B⁵)₂, wherein each B⁵ is independently selected from a C₁-C₄ alkyl, C₂-C₄ alkenyl or C₂-C₄ alkynyl group all of which may optionally be halo-substituted.

Typically, —R² has a formula selected from:

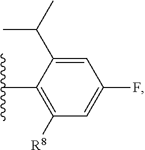

wherein R⁸ is a 5- or 6-membered, optionally substituted heterocyclic or aromatic group. In one embodiment, the optional substituents on the heterocyclic or aromatic group of R⁸ are independently selected from halo, —OH, —NH₂, —CN, —NO₂, —B⁶, —CH₂B⁶, —OB⁶, —OCH₂B⁶, —NHB⁶, —N(B⁶)₂, —CONH₂, —CONHB⁶, —CON(B⁶)₂, —NHCOB⁶, —NB⁶COB⁶, or —B⁶⁶—;
wherein each B⁶ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁶ together with the nitrogen atom to which they are attached may form a 4- to 6-membered heterocyclic group containing one or two ring heteroatoms N and/or O, wherein any B⁶ may optionally be halo-substituted and/or substituted with one or two substituents independently selected from —OH, —NH₂, —B⁶⁷, —OB⁶⁷, —NHB⁶⁷ or —N(B⁶⁷)
wherein each B⁶⁶ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁶⁷, —OB⁶⁷, —NHB⁶⁷ or —N(B⁶⁷)₂; and
wherein each B⁶⁷ is independently selected from a C₁-C₃ alkyl or C₁-C₃ haloalkyl group.

Typically, any divalent group —B⁶⁶— forms a 4- to 6-membered fused ring. Typically, the optional substituents on the heterocyclic or aromatic group of R⁸ are independently selected from halo, —OH, —NH₂, —CN, —NO₂, —B⁶, —OB⁶, —NHB⁶ or —N(B⁶)₂, wherein each B⁶ is independently selected from a C₁-C₄ alkyl, C₂-C₄ alkenyl or C₂-C₄ alkynyl group all of which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. The further substituents on the α-substituted parent cyclic group of R² also include cycloalkyl, cycloalkenyl, non-aromatic heterocyclic, aryl or heteroaryl rings which are fused to the α-substituted parent cyclic group of R². Typically, the cycloalkyl, cycloalkenyl, non-aromatic heterocyclic, aryl or heteroaryl rings are ortho-fused to the α-substituted parent cyclic group of R², i.e. each fused cycloalkyl, cycloalkenyl, non-aromatic heterocyclic, aryl or heteroaryl ring has only two atoms and one bond in common with the α-substituted parent cyclic group of R². Typically, the cycloalkyl, cycloalkenyl, non-aromatic heterocyclic, aryl or heteroaryl rings are ortho-fused to the α-substituted parent cyclic group of R² across the α′,β′ positions.

In one embodiment, —R² has a formula selected from:

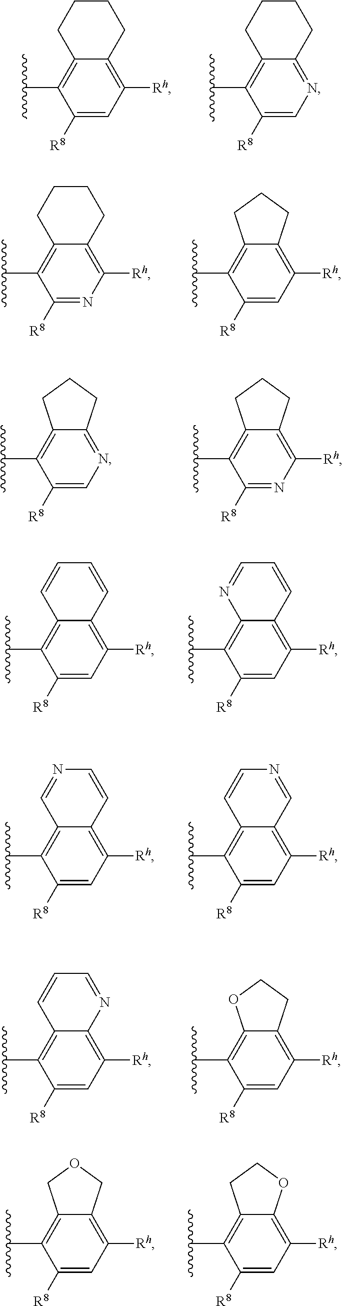

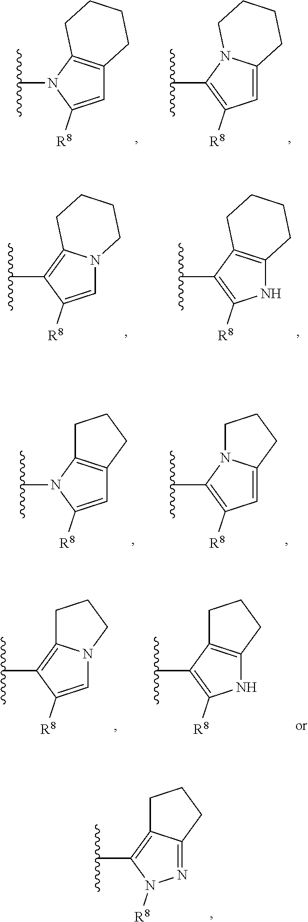

wherein R⁸ is a 5- or 6-membered, optionally substituted heterocyclic or aromatic group, and R^{h} is hydrogen, halo, —OH, —NO₂, —CN, —R^{hh}, —R^{h}, —OR^{hh}, —COR^{hh}, —COOR^{hh}, —CONH₂, —CONHR^{hh}, —CON(R^{hh})₂, —C(═NH)R^{hh}, —C(═NH)NH₂, —C(═NH)NHR^{hh}, —C(═NH)N(R^{hh})₂, —C(═NR^{hh})R^{hh}, —C(═NR^{hh})NHR^{hh}, —C(═NR^{hh})N(R^{hh})₂, —C(═NOH)R^{hh} or —C(═NOR^{hh})R^{hh}, wherein each —R^{hh} is independently selected from C₁-C₄ alkyl, C₁-C₄ haloalkyl, C₃-C₄ cycloalkyl and C₃-C₄ halocycloalkyl, or any two R^{hh} attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{hx} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁷, —CH₂B⁷, —OB⁷, —OCH₂B⁷, —NHB⁷, —N(B⁷)₂, —CONH₂, —CONHB⁷, —CON(B⁷)₂, —NHCOB⁷, —NB⁷COB⁷, or —B⁷⁷—;
wherein each B⁷ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁷ together with the nitrogen atom to which they are attached may form a 4- to 6-membered heterocyclic group containing one or two ring heteroatoms N and/or O, wherein any B⁷ may optionally be halo-substituted and/or substituted with one or two substituents independently selected from —OH, —NH₂, —B⁷⁸, —OB⁷⁸, —NHB⁷⁸ or —N(B⁷⁸)₂;
wherein each B⁷⁷ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁷⁸, —OB⁷⁸, —NHB⁷⁸ or —N(B⁷⁸)₂; and
wherein each B⁷⁸ is independently selected from a C₁-C₃ alkyl or C₁-C₃ haloalkyl group.

Typically, any divalent group —B⁷⁷— forms a 4- to 6-membered fused ring. Typically, R^{h} is hydrogen, halo, —CN, —R^{hh}, —R^{h}, —OR^{hh}, —COR^{hh}, —C(═NOH)R^{hh} or —C(═NOR^{hh})R^{hh}, wherein each —R^{hh} is independently selected from C₁-C₃ alkyl, C₁-C₃ fluoroalkyl, cyclopropyl or fluorocyclopropyl, and wherein —R^{h} is selected from a phenyl, halophenyl or a 5- or 6-membered heteroaryl group, wherein the 5- or 6-membered heteroaryl group is optionally halo substituted. More typically, R^{h} is hydrogen or halo. Typically, the optional substituents on the heterocyclic or aromatic group of R⁸ are independently selected from halo, —OH, —NH₂, —CN, —NO₂, —B⁷, —OB⁷, —NHB⁷ or —N(B⁷)₂, wherein each B⁷ is independently selected from a C₁-C₄ alkyl, C₂-C₄ alkenyl or C₂-C₄ alkynyl group all of which may optionally be halo-substituted.

In one embodiment, —R² has a formula selected from:

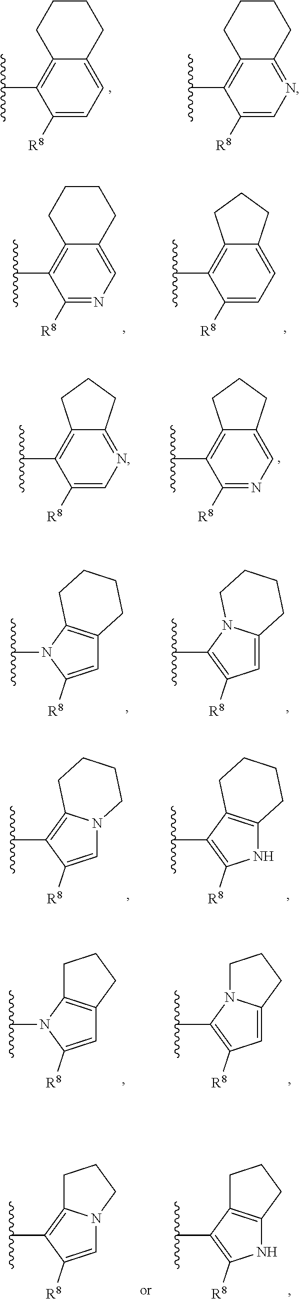

wherein R⁸ is a 5- or 6-membered, optionally substituted heterocyclic or aromatic group. In one embodiment, the optional substituents on the heterocyclic or aromatic group of R⁸ are independently selected from halo, —OH, —NH₂, —CN, —NO₂, —B⁸, —CH₂B⁸, —OB⁸, —OCH₂B⁸, —NHB⁸, —N(B⁸)₂, —CONH₂, —CONHB⁸, —CON(B⁸)₂, —NHCOB⁸, —NB⁸COB⁸, or —B⁸⁸—;
wherein each B⁸ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁸ together with the nitrogen atom to which they are attached may form a 4- to 6-membered heterocyclic group containing one or two ring heteroatoms N and/or O, wherein any B⁸ may optionally be halo-substituted and/or substituted with one or two substituents independently selected from —OH, —NH₂, —B⁸⁹, —OB⁸⁹, —NHB⁸⁹ or —N(B⁸⁹)₂;
wherein each B⁸⁸ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁸⁹, —OB⁸⁹, —NHB⁸⁹ or —N(B⁸⁹)₂; and
wherein each B⁸⁹ is independently selected from a C₁-C₃ alkyl or C₁-C₃ haloalkyl group.

Typically, any divalent group —B⁸⁸— forms a 4- to 6-membered fused ring. Typically, the optional substituents on the heterocyclic or aromatic group are independently selected from halo, —OH, —NH₂, —CN, —NO₂, —B⁸, —OB⁸, —NHB⁸ or —N(B⁸)₂, wherein each B⁸ is independently selected from a C₁-C₄ alkyl, C₂-C₄ alkenyl or C₂-C₄ alkynyl group all of which may optionally be halo-substituted.

Typically, —R² has a formula selected from:

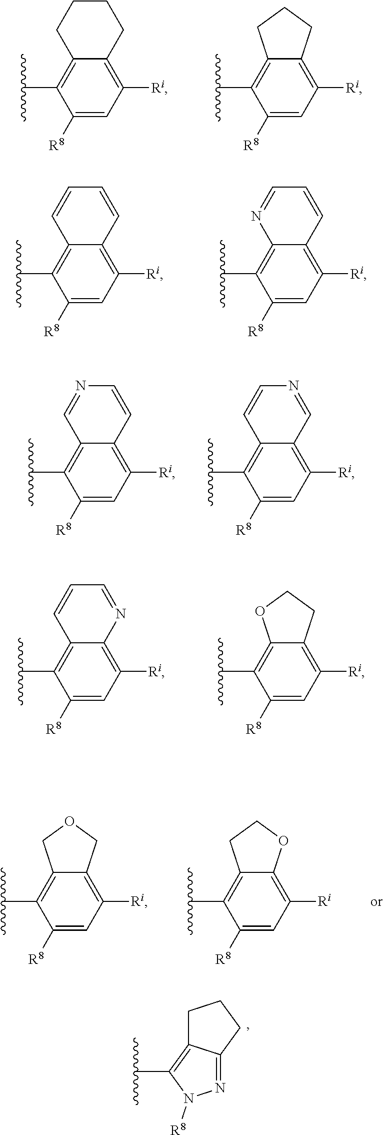

wherein R⁸ is a 5- or 6-membered, optionally substituted heterocyclic or aromatic group, and R¹ is hydrogen, halo, —OH, —NO₂, —CN, —Rⁱⁱ, —R^{ix}, —ORⁱⁱ, —CORⁱⁱ, —COORⁱⁱ, —CONH₂, —CONHRⁱⁱ, —CON(Rⁱⁱ)₂, —C(═NH)Rⁱⁱ, —C(═NH)NH₂, —C(═NH)NHRⁱⁱ, —C(═NH)N(Rⁱⁱ)₂, —C(═NRⁱⁱ)Rⁱⁱ, —C(═NRⁱⁱ)NHRⁱⁱ, —C(═NRⁱⁱ)N(Rⁱⁱ)₂, —C(═NOH)Rⁱⁱ or —C(═NORⁱⁱ)Rⁱⁱ, wherein each —Rⁱⁱ is independently selected from C₁-C₄ alkyl, C₁-C₄ haloalkyl, C₃-C₄ cycloalkyl and C₃-C₄ halocycloalkyl, or any two R¹¹ attached to the same nitrogen atom may, together with the nitrogen atom to which they are attached, form a 3- to 6-membered saturated heterocyclic group, wherein the 3- to 6-membered saturated heterocyclic group is optionally halo substituted, and wherein —R^{ix} is selected from a 3- to 7-membered cyclic group, wherein the 3- to 7-membered cyclic group is optionally halo substituted. In one embodiment, the optional substituents on the heterocyclic or aromatic group of R⁸ are independently selected from halo, —OH, —NH₂, —CN, —NO₂, —B⁹, —CH₂B⁹, —OB⁹, —OCH₂B⁹, —NHB⁹, —N(B⁹)₂, —CONH₂, —CONHB⁹, —CON(B⁹)₂, —NHCOB⁹, —NB⁹COB⁹, or —B⁹⁹—;
wherein each B⁹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁹ together with the nitrogen atom to which they are attached may form a 4- to 6-membered heterocyclic group containing one or two ring heteroatoms N and/or O, wherein any B⁹ may optionally be halo-substituted and/or substituted with one or two substituents independently selected from —OH, —NH₂, —B⁹⁸, —OB⁹⁸, —NHB⁹⁸ or —N(B⁹⁸)₂;
wherein each B⁹⁹ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B⁹⁸, —OB⁹⁸, —NHB⁹⁸ or —N(B⁹⁸)₂; and
wherein each B⁹⁸ is independently selected from a C₁-C₃ alkyl or C₁-C₃ haloalkyl group.

Typically, any divalent group —B⁹⁹— forms a 4- to 6-membered fused ring. Typically, Rⁱ is hydrogen, halo, —CN, C₁-C₃ alkyl, C₁-C₃ haloalkyl, cyclopropyl or halocyclopropyl. Typically, Rⁱ is hydrogen, halo, —CN, —Rⁱⁱ, —R^{ix}, —ORⁱⁱ, —CORⁱⁱ, —C(═NOH)Rⁱⁱ or —C(═NORⁱⁱ)Rⁱⁱ, wherein each —Rⁱⁱ is independently selected from C₁-C₃ alkyl, C₁-C₃ fluoroalkyl, cyclopropyl or fluorocyclopropyl, and wherein —R^{ix} is selected from a phenyl, halophenyl or a 5- or 6-membered heteroaryl group, wherein the 5- or 6-membered heteroaryl group is optionally halo substituted. More typically, Rⁱ is hydrogen or halo. Typically, the optional substituents on the heterocyclic or aromatic group of R⁸ are independently selected from halo, —OH, —NH₂, —CN, —NO₂, —B⁹, —OB⁹, —NHB⁹ or —N(B⁹)₂, wherein each B⁹ is independently selected from a C₁-C₄ alkyl, C₂-C₄ alkenyl or C₂-C₄ alkynyl group all of which may optionally be halo-substituted.

In one embodiment, R² is phenyl or a 5- or 6-membered heteroaryl group (such as phenyl, pyridinyl, pyridazinyl, pyrimidinyl, pyrazinyl, pyrrolyl, pyrazolyl or imidazolyl); wherein:
(i) the phenyl or 5- or 6-membered heteroaryl group is substituted at the α position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted at the α′ position with a substituent selected from —R¹⁴, —OR¹⁴ and —COR¹⁴, wherein R¹⁴ is selected from a C₁-C₆ alkyl, C₂-C₆ alkenyl, C₂-C₆ alkynyl or C₂-C₆ cyclic group and wherein R¹⁴ is optionally substituted with one or more halo groups; and
optionally the phenyl or 5- or 6-membered heteroaryl group is further substituted (typically with one, two or three substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or
(ii) the phenyl or 5- or 6-membered heteroaryl group is substituted with a cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and optionally the phenyl or 5- or 6-membered heteroaryl group is further substituted (typically with one or two substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or (iii) the phenyl or 5- or 6-membered heteroaryl group is substituted with a first cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and the phenyl or 5- or 6-membered heteroaryl group is substituted with a second cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and optionally the phenyl group is further substituted (typically with a substituent selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or (iv) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, —CN, —R¹³, —OR¹³, —N(R¹³)₂, —C≡CR¹³, —R¹²—R¹³, —R¹²—OR¹³, —R¹²—N(R¹³)₂, —R¹²—C≡CR¹³, —O—R¹²—CN, —O—R¹²—R¹³, —O—R¹²—OR¹³, —O—R¹²—N(R¹³)₂ or —O—R¹²—C≡CR¹³, and wherein a ring atom of the monovalent heterocyclic or aromatic group is directly attached to the α-ring atom of the parent phenyl or 5- or 6-membered heteroaryl group; wherein R¹² is independently selected from a C₁-C₃ alkylene or C₁-C₃ haloalkylene group; and R¹³ is independently selected from hydrogen or a C₁-C₄ alkyl, C₁-C₄ haloalkyl or 3- to 6-membered cyclic group (such as a C₃-C₆ cycloalkyl, phenyl, or 4- to 6-membered saturated heterocyclic group), wherein the 3- to 6-membered cyclic group may optionally be substituted with one or more halo, methyl or halomethyl groups; and
optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted (typically with one, two or three substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group); or (v) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, —CN, —R¹³, —OR¹³, —N(R¹³)₂, —C≡CR¹³, —R¹²—CN, —R¹²—R¹³, —R¹²—OR¹³, —R¹²—N(R¹³)₂, —R¹²—C≡CR¹³, —O—R¹²—CN, —O—R¹²—R¹³, —O—R¹²—OR¹³, —O—R¹²—N(R¹³)₂ or —O—R¹²—C≡CR¹³, and wherein a ring atom of the monovalent heterocyclic or aromatic group is directly attached to the α-ring atom of the parent phenyl or 5- or 6-membered heteroaryl group; wherein R¹² is independently selected from a C₁-C₃ alkylene or C₁-C₃ haloalkylene group; and R¹³ is independently selected from hydrogen or a C₁-C₄ alkyl, C₁-C₄ haloalkyl or 3- to 6-membered cyclic group (such as a C₃-C₆ cycloalkyl, phenyl, or 4- to 6-membered saturated heterocyclic group), wherein the 3- to 6-membered cyclic group may optionally be substituted with one or more halo, methyl or halomethyl groups; and
optionally the phenyl or 5- or 6-membered heteroaryl group is further substituted with a cycloalkyl, cycloalkenyl, non-aromatic heterocyclic, aryl or heteroaryl ring which is fused to the parent phenyl or s- or 6-membered heteroaryl group across the α′,β′ positions and which is optionally substituted with one or more halo groups; and
optionally the phenyl or 5- or 6-membered heteroaryl group is further substituted (typically with one or two substituents independently selected from halo, —NO₂, —CN, C₁-C₄ alkyl, C₁-C₄ haloalkyl, a 3- to 5-membered cyclic group (such as a 5-membered heteroaryl group), a 3- to 5-membered halocyclic group (such as a 5-membered haloheteroaryl group), —COR¹⁵, —COOR¹⁵, —CONH₂, —CONHR¹⁵, —CON(R¹⁵)₂, —C(═NOH)R¹⁵ or —C(═NOR¹⁵)R¹⁵, wherein each —R¹⁵ is independently selected from a C₁-C₄ alkyl or C₁-C₄ haloalkyl group).

In the embodiment directly above, where a group or moiety is optionally substituted with one or more halo groups, it may be substituted for example with one, two, three, four, five or six halo groups.

In one aspect of any of the above embodiments, R² contains from 10 to 50 atoms other than hydrogen or halogen. More typically, R² contains from 10 to 40 atoms other than hydrogen or halogen. More typically, R² contains from 10 to 35 atoms other than hydrogen or halogen. More typically still, R² contains from 10 to 30 or from 12 to 30 atoms other than hydrogen or halogen. Yet more typically, R² contains from 10 to 25 or from 12 to 25 atoms other than hydrogen or halogen.

In one aspect of any of the above embodiments, the compound of formula (I) has a molecular weight of from 250 to 2000 Da. Typically, the compound of formula (I) has a molecular weight of from 280 to 900 Da. More typically, the compound of formula (I) has a molecular weight of from 290 to 600 Da.

A second aspect of the invention provides a compound selected from the group consisting of:

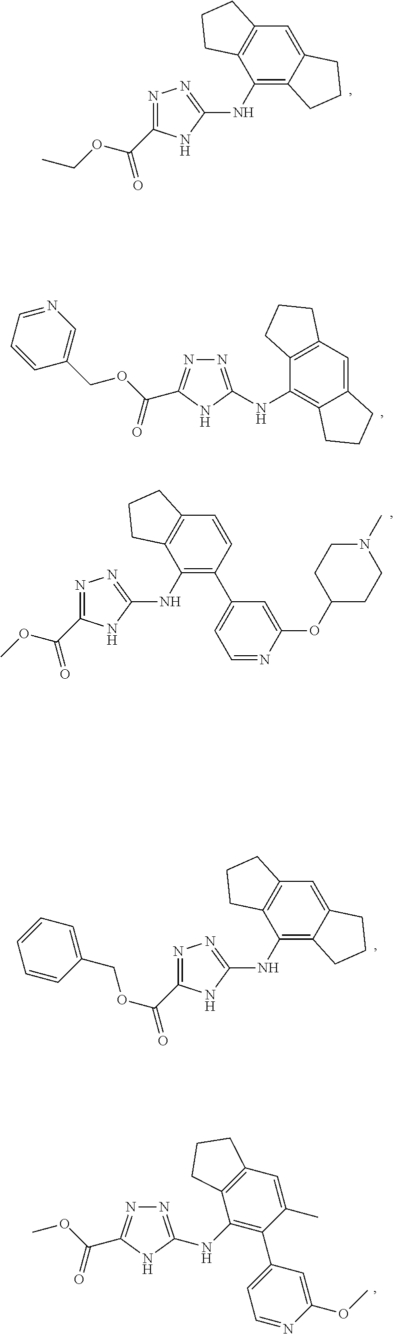

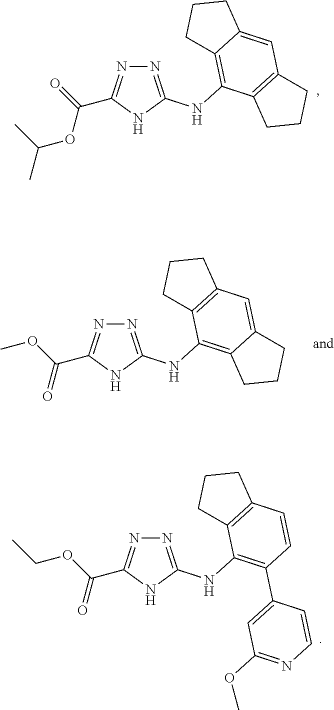 [SMILES: CC(C)OC(=O)c1nnc(Nc2c3c(cc4c2CCC4)CCC3)[nH]1 ; COC(=O)c1nnc(Nc2c3c(cc4c2CCC4)CCC3)[nH]1 ; CCOC(=O)c1nnc(Nc2c(-c3ccnc(OC)c3)ccc3c2CCC3)[nH]1]

A third aspect of the invention provides a pharmaceutically acceptable salt, solvate or prodrug of any compound of the first or second aspect of the invention.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p s 0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

Genetic diseases in which a role for NLRP3 has been suggested include sickle cell disease (Vogel et al., Blood, 130(Suppl 1): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur J Immunol, 40: 595-653, 2010). In particular, NLRP3 mutations have been found to be responsible for a set of rare autoinflammatory diseases known as CAPS (Ozaki et al., J Inflammation Research, 8: 15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome, Coeliac disease (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; Pontillo et al., Autoimmunity, 43(8): 583-589, 2010; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of respiratory and lung diseases including chronic obstructive pulmonary disorder (COPD), asthma (including steroid-resistant asthma and eosinophilic asthma), bronchitis, asbestosis, volcanic ash induced inflammation, and silicosis (Cassel et al., Proceedings of the National Academy of Sciences, 105(26): 9035-9040, 2008; Chen et al., ERJ Open Research, 4: 00130-2017, 2018; Chen et al., Toxicological Sciences, 170(2): 462-475, 2019; Damby et al., Front Immun, 8: 2000, 2018; De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014; Cheng et al., Autophagy, 1-13, 2020; Couturier et al., J Neuroinflamm, 13: 20, 2016; and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018; Hong et al., Neural Plasticity, 2018: 8, 2018; Ye et al., Experimental Neurology, 292: 46-55, 2017), general anesthesia neuroinflammation (Fan et al., Front Cell Neurosci, 12: 426, 2018), sepsis-associated encephalopathy (SAE) (Fu et al., Inflammation, 42(1): 306-318, 2019), perioperative neurocognitive disorders including postoperative cognitive dysfunction (POCD) (Fan et al., Front Cell Neurosci, 12: 426, 2018; and Fu et al., International Immunopharmacology, 82: 106317, 2020), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018; and Chen et al., Brain Research, 1710: 163-172, 2019).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis (Chen et al., Journal of the American Heart Association, 6(9): e006347, 2017; and Chen et al., Biochem Biophys Res Commun, 495(1): 382-387, 2018), myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vase Biol, 37(4): 694-706, 2017), cardiac injury induced by metabolic dysfunction (Pavillard et al., Oncotarget, 8(59): 99740-99756, 2017; and Zhang et al., Biochimica et Biophysica Acta, 1863(6): 1556-1567, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJM0a1707914, 2017).

Other diseases, disorders and conditions in which NLRP3 has been shown to be involved include:
ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);
liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure including acute liver failure (Wang et al., Hepatol Res, 48(3): E194-E202, 2018);
kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), obesity related glomerulopathy (Zhao et al., Mediators of Inflammation, article 3172647, 2019), acute kidney injury (Zhang et al., Diabetes, Metabolic Syndrome and Obesity: Targets and Therapy, 12: 1297-1309, 2019), and renal hypertension (Krishnan et al., Br J Pharmacol, 173(4): 752-65, 2016; Krishnan et al., Cardiovasc Res, 115(4): 776-787, 2019; Dinh et al., Aging, 9(6): 1595-1606, 2017);
conditions associated with diabetes including diabetic encephalopathy (Zhai et al., Molecules, 23(3): 522, 2018), diabetic retinopathy (Zhang et al., Cell Death Dis, 8(7): e2941, 2017), diabetic nephropathy (also called diabetic kidney disease) (Chen et al., BMC Complementary and Alternative Medicine, 18: 192, 2018), and diabetic hypoadiponectinemia (Zhang et al., Biochimica et Biophysica Acta (BBA)—Molecular Basis of Disease, 1863(6): 1556-1567, 2017);
inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);
inflammatory reactions in the joints (Braddock et al., Nat Rev Drug Disc, 3: 1-10, 2004) and osteoarthritis (Jin et al., PNAS, 108(36): 14867-14872, 2011);
conditions associated with arthritis including arthritic fever (Verma, Linköping University Medical Dissertations, No. 1250, 2011);
amyotrophic lateral sclerosis (Gugliandolo et al., Inflammation, 41(1): 93-103, 2018);
cystic fibrosis (Iannitti et al., Nat Commun, 7: 10791, 2016);
stroke (Walsh et al., Nature Reviews, 15: 84-97, 2014; Ye et al., Experimental Neurology, 292: 46-55, 2017);
headaches including migraine (He et al., Journal of Neuroinflammation, 16: 78, 2019);
chronic kidney disease (Granata et al., PLoS One, 10(3): e0122272, 2015);
Sjögren's syndrome (Vakrakou et al., Journal of Autoimmunity, 91: 23-33, 2018);
graft-versus-host disease (Takahashi et al., Scientific Reports, 7: 13097, 2017);
sickle cell disease (Vogel et al., Blood, 130(Suppl 1): 2234, 2017); and
colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), burn healing (Chakraborty et al., Exp Dermatol, 27(1): 71-79, 2018), pain including allodynia, multiple sclerosis-associated neuropathic pain (Khan et al., Inflammopharmacology, 26(1): 77-86, 2018), chronic pelvic pain (Zhang et al., Prostate, 79(12): 1439-1449, 2019) and cancer-induced bone pain (Chen et al., Pharmacological Research, 147: 104339, 2019), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus, including methicillin-resistant Staphylococcus aureus (MRSA) (Cohen et al., Cell Reports, 22(9): 2431-2441, 2018; and Robinson et al., JCI Insight, 3(7): e97470, 2018), Mycobacterium tuberculosis (TB) (Subbarao et al., Scientific Reports, 10: 3709, 2020), Bacillus cereus (Mathur et al., Nat Microbiol, 4: 362-374, 2019), Salmonella typhimurium (Diamond et al., Sci Rep, 7(1): 6861, 2017), and group A Streptococcus (LaRock et al., Science Immunology, 1(2): eaah3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), leishmania (Novais et al., PLoS Pathogens, 13(2): e1006196, 2017), and plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012). NLRP3 activity has also been associated with increased susceptibility to viral infection such as by the human immunodeficiency virus (HIV) (Pontillo et al., J Aquir Immune Defic Syndr, 54(3): 236-240, 2010). An increased risk for early mortality amongst patients co-infected with HIV and Mycobacterium tuberculosis (TB) has also been associated with NLRP3 activity (Ravimohan et al., Open Forum Infectious Diseases, 5(5): ofy075, 2018).

NLRP3 has been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016), and NLRP3 has been shown to suppress NK cell-mediated control of carcinogenesis and metastases (Chow et al., Cancer Res, 72(22): 5721-32, 2012). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017; and Chen et al., Cellular and Molecular Life Sciences, 75: 2045-2058, 2018). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, any of the diseases, disorders or conditions listed above may be treated or prevented in accordance with the fifth, sixth, seventh, eighth, ninth or tenth aspect of the present invention. Particular examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease including paediatric Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis, a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, squamous cell carcinoma of the head and neck, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus (including MRSA), Helicobacter pylori, Bacillus anthracis, Bacillus cereus, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, Uropathogenic Escherichia coli (UPEC) or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from Schistosoma, roundworms, tapeworms or flukes), prion infections, and co-infections with any of the aforementioned (e.g. with HIV and Mycobacterium tuberculosis);

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, perioperative neurocognitive disorder, postoperative cognitive dysfunction, early brain injury, traumatic brain injury, cerebral ischemia-reperfusion injury, stroke, general anesthesia neuroinflammation and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, metabolism induced cardiac injury, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, volcanic ash induced inflammation, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure including acute liver failure;

(x) renal diseases including chronic kidney disease, oxalate nephropathy, nephrocalcinosis, glomerulonephritis, diabetic nephropathy, obesity related glomerulopathy, kidney fibrosis including chronic crystal nephropathy, acute renal failure, acute kidney injury, and renal hypertension;

(xi) ocular diseases including those of the ocular epithelium, age-related macular degeneration (AMD) (dry and wet), Sjögren's syndrome, uveitis, corneal infection, diabetic retinopathy, optic nerve damage, dry eye, and glaucoma;

(xii) skin diseases including dermatitis such as contact dermatitis and atopic dermatitis, contact hypersensitivity, psoriasis, sunburn, skin lesions, hidradenitis suppurativa (HS), other cyst-causing skin diseases, pyoderma gangrenosum, and acne vulgaris including acne conglobata;

(xiii) lymphatic conditions such as lymphangitis and Castleman's disease;

(xiv) psychological disorders such as depression and psychological stress;

(xv) graft versus host disease;

(xvi) pain such as pelvic pain, hyperalgesia, allodynia including mechanical allodynia, neuropathic pain including multiple sclerosis-associated neuropathic pain, and cancer-induced bone pain;

(xvii) conditions associated with diabetes including diabetic encephalopathy, diabetic retinopathy, diabetic nephropathy, diabetic vascular endothelial dysfunction, and diabetic hypoadiponectinemia;

(xviii) conditions associated with arthritis including arthritic fever;

(xix) headache including cluster headaches, idiopathic intracranial hypertension, migraine, low pressure headaches (e.g. post-lumbar puncture), Short-Lasting Unilateral Neuralgiform Headache With Conjunctival Injection and Tearing (SUNCT), and tension-type headaches;

(xx) wounds and burns, including skin wounds and skin burns; and

(xxi) any disease where an individual has been determined to carry a germline or somatic non-silent mutation in NLRP3.

In one embodiment, the disease, disorder or condition is selected from:

(i) inflammation;

(ii) an auto-immune disease;

(iii) cancer;

(iv) an infection;

(v) a central nervous system disease;

(vi) a metabolic disease;

(vii) a cardiovascular disease;

(viii) a respiratory disease;

(ix) a liver disease;

(x) a renal disease;

(xi) an ocular disease;

(xii) a skin disease;

(xiii) a lymphatic condition;

(xiv) a psychological disorder;

(xv) graft versus host disease;

(xvi) allodynia;

(xvii) a condition associated with diabetes; and

(xviii) any disease where an individual has been determined to carry a germline or somatic non-silent mutation in NLRP3.

In another embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxxiii) transient ischemic attack;

(xxxiv) ulcerative colitis; or

(xxxv) Valosin Containing Protein disease.

In another embodiment of the fifth, sixth, seventh, eighth, ninth or tenth aspect of the present invention, the treatment or prevention comprises a reduction in susceptibility to viral infection. For instance, the treatment or prevention may comprise a reduction in susceptibility to HIV infection.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis;

(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, Legionella, Lyme disease, influenza A, Epstein-Barr virus infection, viral encephalitis/aseptic meningitis, or pelvic inflammatory disease;

(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;

(xv) a cancer, including those cancers listed above;

(xvi) a burn, wound, trauma, haemorrhage or stroke;

(xvii) radiation exposure;

(xviii) a metabolic disease such as type 2 diabetes (T2D), atherosclerosis, obesity, gout or pseudo-gout; and/or

(xix) pain such as inflammatory hyperalgesia, pelvic pain, allodynia, neuropathic pain, or cancer-induced bone pain.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

As will be understood, where the further active agent is a small chemical entity, any reference to a specific small chemical entity below is to be understood to encompass all salt, hydrate, solvate, polymorphic and prodrug forms of the specific small chemical entity. Similarly, where the further active agent is a biologic such as a monoclonal antibody, any reference to a specific biologic below is to be understood to encompass all biosimilars thereof.

In some embodiments, the one or more chemotherapeutic agents are selected from abiraterone acetate, altretamine, amsacrine, anhydrovinblastine, auristatin, azacitidine, 5-azacytidine, azathioprine, adriamycin, bexarotene, bicalutamide, BMS 184476, bleomycin, bortezomib, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cladribine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, ixazomib, lenalidomide, lenalidomide-dexamethasone, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, revlimid, rhizoxin, sertenef, streptozocin, stramustine phosphate, tretinoin, tasonermin, taxol, topotecan, tamoxifen, teniposide, taxane, tegafur/uracil, thalidomide, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), Type I interferon ligands such as interferon alpha and interferon beta, Type I interferon mimetics, Type II interferon ligands such as interferon gamma, Type II interferon mimetics, interferon inducible protein (IP-10), kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), cytokines (including interleukins, such as interleukin-1, interleukin-2, interleukin-5, interleukin-10, interleukin-12, and interleukin-33), interleukin-1 ligands and mimetics (such as rilonacept, anakinra, and anakinra-dexamethasone), interleukin-2 ligands and mimetics, interleukin-5 ligands and mimetics, interleukin-10 ligands and mimetics, interleukin-12 ligands and mimetics, and/or interleukin-33 ligands and mimetics.

In some embodiments, the one or more antibodies may comprise one or more monoclonal antibodies. In some embodiments, the one or more antibodies are anti-TNFα and/or anti-IL-6 antibodies, in particular anti-TNFα and/or anti-IL-6 monoclonal antibodies. In some embodiments, the one or more antibodies are selected from abatacept, abciximab, adalimumab, alemtuzumab, atezolizumab, atlizumab, avelumab, basiliximab, belimumab, benralizumab, bevacizumab, bretuximab vedotin, brodalumab, canakinumab, cetuximab, ceertolizumab pegol, daclizumab, denosumab, dupilumab, durvalumab, eculizumab, efalizumab, elotuzumab, gemtuzumab, golimumab, guselkumab, ibritumomab tiuxetan, infliximab, ipilimumab, ixekizumab, mepolizumab, muromonab-CD3, natalizumab, nivolumab, ofatumumab, omalizumab, palivizumab, panitumuab, pembrolizumab, ranibizumab, reslizumab, risankizumab, rituximab, sarilumab, secukinumab, siltuximab, tildrakizumab, tocilizumab, tositumomab, trastuzumab, and/or ustekinumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from thalidomide, lenalidomide, endostatin, angiogenin inhibitors, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a Vinca alkaloid, a podophyllotoxin and/or a taxane. In some embodiments, the one or more Vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides (CDNs), such as c-di-AMP, c-di-GMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃). In some embodiments, the one or more STING agonists are selected from BMS-986301, MK-1454, ADU-S100, a diABZI, 3′3′-cGAMP, and/or 2′3′-cGAMP.

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TLiA, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDRooi (PD1), BMS-986016, MGA271, lirilumab, IPH2201, emactuzumab, INCBo24360, galunisertib, ulocuplumab, BKT140, bavituximab, CC-90002, bevacizumab, and/or MNRP1685A.

In some embodiments, the one or more immunomodulatory agents may comprise a complement pathway modulator. Complement pathway modulators modulate the complement activation pathway. Complement pathway modulators may act to block action of the C3 and/or C3a and/or C3aR1 receptor, or may act to block action of the C5 and/or C5a and/or C5aR1 receptor. In some embodiments, the complement pathway modulator is a C5 complement pathway modulator and may be selected from eculizumab, ravulizumab (ALXN1210), ABP959, RA101495, tesidolumab (LFG316), zimura, crovalimab (RO7112689), pozelimab (REGN3918), GNR-045, SOBI005, and/or coversin. In some embodiments, the complement pathway modulator is a C5a complement pathway modulator and may be selected from cemdisiran (ALN-CC5), IFX-1, IFX-2, IFX-3, and/or olendalizumab (ALXN1007). In some embodiments, the complement pathway modulator is a C5aR1 complement pathway modulator and may be selected from ALS-205, MOR-210/TJ210, DF2593A, DF3016A, DF2593A, avacopan (CCX168), and/or IPH5401.

In some embodiments, the one or more immunomodulatory agents may comprise an anti-TNFα agent. In some embodiments, the anti-TNFα agent may be an antibody or an antigen-binding fragment thereof, a fusion protein, a soluble TNFα receptor (e.g. a soluble TNFR1 or soluble TNFR2), an inhibitory nucleic acid, or a small molecule TNFα antagonist. In some embodiments, the inhibitory nucleic acid may be a ribozyme, a small hairpin RNA, a small interfering RNA, an antisense nucleic acid, or an aptamer.

In some embodiments, the anti-TNFα agent is selected from adalimumab, certolizumab pegol, etanercept, golimumab, infliximab, CDP571, and biosimilars thereof (such as adalimumab-adbm, adalimumab-adaz, adalimumab-atto, etanercept-szzs, infliximab-abda and infliximab-dyyb).

In some embodiments, the one or more immunomodulatory agents may comprise azithromycin, clarithromycin, erythromycin, levofloxacin and/or roxithromycin.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents; anti-inflammatory agents (including non-steroidal anti-inflammatory agents; small molecule anti-inflammatory agents (such as colchicine); and anti-inflammatory biologics that target for example TNF, IL-5, IL-6, IL-17 or IL-33); JAK inhibitors; phosphodiesterase inhibitors; CAR T therapies; anti-psoriatic agents (including anthralin and its derivatives); vitamins and vitamin-derivatives (including retinoids, and VDR receptor ligands); steroids; corticosteroids; glucocorticoids (such as dexamethasone, prednisone and triamcinolone acetonide); ion channel blockers (including potassium channel blockers); immune system regulators (including cyclosporin, FK 506, and glucocorticoids); lutenizing hormone releasing hormone agonists (such as leuprolidine, goserelin, triptorelin, histrelin, bicalutamide, flutamide and/or nilutamide); hormones (including estrogen); and/or uric acid lowering agents (such as allopurinol).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES—COMPOUND SYNTHESIS

All solvents, reagents and compounds were purchased and used without further purification unless stated otherwise.

### Abbreviations

AcOH acetic acid
app apparent
aq aqueous
B²Pin₂ bis(pinacolato)diboron
Boc tert-butyloxycarbonyl
br broad
Cbz carboxybenzyl
CDI 1,1-carbonyl-diimidazole
conc concentrated
m-CPBA 3-chlorobenzoperoxoic acid
d doublet
DCM dichloromethane
dd double doublet
DBU 1,8-diazabicyclo[5.4.0]undec-7-ene
DIPEA diisopropylethylamine
DMA N,N-dimethylacetamide
DMAP N,N-dimethylpyridin-4-amine
DMF N,N-dimethylformamide
DMSO dimethylsulfoxide
dt/td double triplet/triple doublet
(ES+)/(ES−) electrospray ionization, positive/negative mode
Et ethyl
EtOAc ethyl acetate
EtOH ethanol
h hour(s)
HATU 1-[bis(dimethylamino)methylene]-1H-1,2,3-triazolo[4,5-b]pyridinium 3-oxid hexafluorophosphate
HMBC-NMR Heteronuclear multiple-bond correlation NMR
HPLC high performance liquid chromatography (reverse phase)
LC liquid chromatography
LiHMDS Lithium bis(trimethylsilyl)amide
m multiplet
(M+H)+ protonated molecular ion
Me methyl
MeCN acetonitrile
MeOH methanol
MHz megahertz
min minute(s)
Ms methanesulfonyl
MS mass spectrometry
MTBE/TBME methyl tert-butyl ether
m/z mass-to-charge ratio
NBS 1-bromopyrrolidine-2,5-dione
NCS 1-chloropyrrolidine-2,5-dione
NMP N-methylpyrrolidine
NMR nuclear magnetic resonance (spectroscopy)
Oxone potassium peroxymonosulfate
p pentuplet
Pd-175 [tBuBrettPhosPd(allyl)]OTf: (allyl(2-di-tert-butylphosphino-2′,4′,6′-triisopropyl-3,6-dimethoxy-1,1′-biphenyl)palladium(II) triflate) from Johnson Matthey
Pd(dba)₂ bis(dibenzylideneacetone)palladium(0)
Pd₂(dba)₃ tris(dibenzylideneacetone)dipalladium(0)
Pd(dppf)Cl₂ [1,1′-bis(diphenylphosphino)ferrocene]dichloropalladium(II)
PE petroleum ether
Ph phenyl
PMB 4-methoxybenzyl
prep-HPLC preparative-high performance liquid chromatography
prep-TLC preparative-thin layer chromatography
q quartet
RP reverse phase
RT room temperature
s singlet
sat saturated
SCX solid supported cation exchange (resin)
SEM 2-(trimethylsilyl)ethyoxy methyl
sept septuplet
t triplet
TBAF tetrabutylammonium fluoride
TEA triethylamine
TFA trifluoroacetic acid
THF tetrahydrofuran
TLC thin layer chromatography
TMS trimethylsilyl
XantPhos (9,9-dimethyl-9H-xanthene-4,5-diyl)bis(diphenylphosphine)
XPhos 2-dicyclohexylphosphino-2′,4′,6′-triisopropylbiphenyl

Experimental Methods

Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker Avance III spectrometer at 400 MHz fitted with a BBO 5 mm liquid probe,
a Bruker 400 MHz spectrometer using ICON-NMR, under TopSpin program control,
a Bruker Avance III HD spectrometer at 500 MHz, equipped with a Bruker 5 mm SmartProbe™,
an Agilent VNMRS 300 instrument fitted with a 7.05 Tesla magnet from Oxford instruments, indirect detection probe and direct drive console including PFG module, or
an Agilent MercuryPlus 300 instrument fitted with a 7.05 Tesla magnet from Oxford instruments, 4 nuclei auto-switchable probe and Mercury plus console.

LC-MS

LC-MS Methods: Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃—H₂O in water (v/v); B: acetonitrile. Column: Kinetex EVO C18 2.1×30 mm, 5 μm.

Preparative Reversed Phase HPLC General Methods

Acidic prep HPLC (x-y % MeCN in water): Waters X-Select CSH column C18, 5 μm (19×50 mm), flow rate 28 mL min-1 eluting with a H₂O-MeCN gradient containing 0.1% v/v formic acid over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN. Acidic prep HPLC (x-y % MeOH in water): Waters X-Select CSH column C18, 5 μm (19×50 mm), flow rate 28 mL min-1 eluting with a 10 mM aq formic acid-MeOH gradient over 7.5 min using UV detection at 254 nm. Gradient information: 0.0-1.5 min, x % MeOH; 1.5-6.8 min, ramped from x % MeOH to y % MeOH; 6.8-6.9 min, ramped from y % MeOH to 95% MeOH; 6.9-7.5 min, held at 95% MeOH.

Basic prep HPLC (x-y % MeCN in water): Waters X-Bridge Prep column C18, 5 μm (19×50 mm), flow rate 28 mL min-1 eluting with a 10 mM NH₄HCO₃-MeCN gradient over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

### Synthesis of Intermediates

### Intermediate A1: ethyl 5-bromo-1-((2-(trimethylsilyl)ethoxy)methyl)-1H-1,2,4-triazole-3-carboxylate

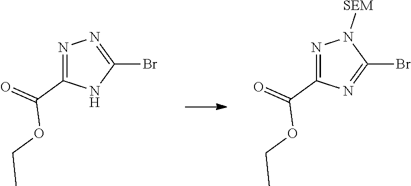 [SMILES: CCOC(=O)c1nnc(Br)[nH]1 ; CCOC(=O)c1nc(Br)n(COCC[Si](C)(C)C)n1]

SEMCl (1.815 mL, 10.25 mmol) was added to K₂CO₃ (2.64 g, 19.09 mmol) and ethyl 5-bromo-4H-1,2,4-triazole-3-carboxylate (2 g, 9.09 mmol) in MeCN (20 mL) at 0° C. The mixture was warmed to RT, stirred for 24 h and then diluted with water (50 mL) and EtOAc (50 mL). The layers were separated and the aqueous layer was extracted with EtOAc (2×30 mL) and the combined organics were dried (MgSO₄) and concentrated in vacuo. The crude was purified by chromatography on silica gel (120 g column, 0-20% EtOAc/isohexane) to afford the title compound (1.09 g, 33% yield) as a thick colourless oil.

LCMS m/z 319.9/321.9 (M-Et+H)⁺ (ES⁺).

¹H NMR (DMSO-d₆) δ 5.76 (s, 2H), 4.39 (q, J=7.1 Hz, 2H), 3.72-3.54 (m, 2H), 1.33 (t, J=7.1 Hz, 3H), 0.86-0.81 (m, 2H), −0.06 (s, 9H).

The following intermediate was synthesised following the general procedure for Intermediate A1:

| Intermediate | Structure | ¹H NMR |
|---|---|---|
| A2 | 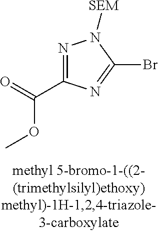 [SMILES: COC(=O)c1nc(Br)n(COCC[Si](C)(C)C)n1] | ¹H NMR (CDC1₃) δ 5.86 (s, 2H), 4.01 (s, 3H), 3.70-3.64 (m, 2H), 0.95-0.90 (m, 2H), −0.02 (s, 9H). |

### Intermediate B1: ethyl 5-((1,2,3,5,6,7-hexahydro-s-indacen-4-yl)amino)-1-((2-(trimethylsilyl)ethoxy)methyl)-1H-1,2,4-triazole-3-carboxylate

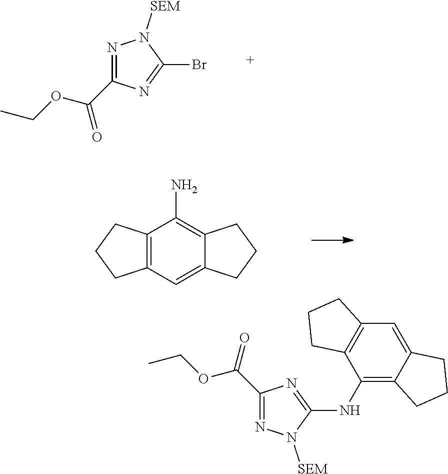 [SMILES: CCOC(=O)c1nc(Br)n(COCC[Si](C)(C)C)n1 ; Nc1c2c(cc3c1CCC3)CCC2 ; CCOC(=O)c1nc(Nc2c3c(cc4c2CCC4)CCC3)n(COCC[Si](C)(C)C)n1]

A mixture of ethyl 5-bromo-1-((2-(trimethylsilyl)ethoxy)methyl)-1H-1,2,4-triazole-3-carboxylate (Intermediate A1) (487.5 mg, 1.392 mmol), K₂CO₃ (308 mg, 2.227 mmol), Pd-175 (109 mg, 0.139 mmol) and 1,2,3,5,6,7-hexahydro-s-indacen-4-amine (241 mg, 1.392 mmol) in 1,4-dioxane (28 mL) was heated to 60° C. under N₂ overnight. The reaction was diluted with EtOAc (so mL), washed with water (so mL) and NH₄Cl (50 mL). The organic phase was separated, dried (phase separator) and concentrated in vacuo. The crude product was purified by chromatography on silica gel (12 g column, 0-50% EtOAc/isohexane) to afford the title compound (267.8 mg, 39% yield) as a yellow oil.

LCMS m/z 443.3 (M+H)⁺ (ES⁺).

¹H NMR (DMSO-d₆) δ 8.29 (s, 1H), 6.87 (s, 1H), 5.59 (s, 2H), 4.34 (q, J=7.1 Hz, 2H), 3.63-3.58 (m, 2H), 2.80 (t, J=7.4 Hz, 4H), 2.66 (t, J=7.4 Hz, 4H), 1.94 (p, J=7.5 Hz, 4H), 1.31 (t, J=7.1 Hz, 3H), 0.86-0.80 (m, 2H), −0.05 (s, 9H).

The following intermediate was synthesised following the general procedure for Intermediate B1:

| Intermediate | Structure | ¹H NMR | LCMS |
|---|---|---|---|
| B2 | 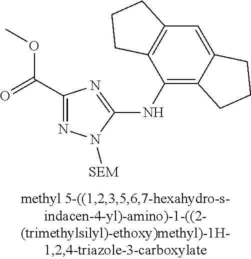 [SMILES: COC(=O)c1nc(Nc2c3c(cc4c2CCC4)CCC3)n(COCC[Si](C)(C)C)n1] | ¹H NMR (DMSO-d₆) δ 8.26 (s, 1H), 6.86 (s, 1H), 5.58 (s, 2H), 3.87 (s, 3H), 3.60 (t, J = 8.0 Hz, 2H), 2.80 (t, J = 7.4 Hz, 4H), 2.65 (t, J = 7.3 Hz, 4H), 1.94 (p, J = 7.4 Hz, 4H), 0.83 (t, J = 8.0 Hz, 2H), −0.06 (s, 9H). | m/z 429.3 (M + H)⁺ (ES⁺) |

### Intermediate C1: sodium 5-((1,2,3,5,6,7-hexahydro-s-indacen-4-yl)amino)-4H-1,2,4-triazole-3-carboxylate

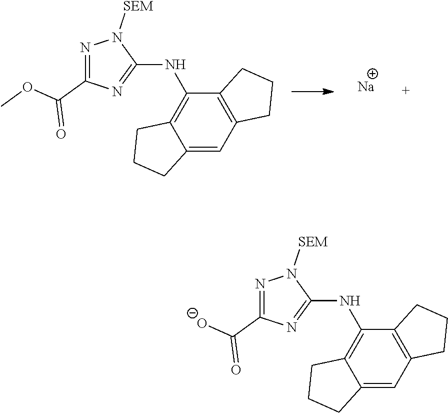 [SMILES: COC(=O)c1nc(Nc2c3c(cc4c2CCC4)CCC3)n(COCC[Si](C)(C)C)n1 ; [Na+] ; C[Si](C)(C)CCOCn1nc(C(=O)[O-])nc1Nc1c2c(cc3c1CCC3)CCC2]

2 M aq NaOH (0.700 mL, 1.400 mmol) was added to methyl 5-((1,2,3,5,6,7-hexahydro-s-indacen-4-yl)amino)-1-((2-(trimethylsilyl)ethoxy)methyl)-1H-1,2,4-triazole-3-carboxylate (Intermediate B²) (0.6 g, 1.40 mmol) in THF (8 mL) and the reaction stirred at RT for 1 h. A further portion of NaOH (0.700 mL, 1.40 mmol) was added and the reaction stirred for 1 h at RT and concentrated in vacuo to afford the crude title compound which was used without further purification.

### Preparation of Examples

### Example 1: ethyl 5-((1,2,3,5,6,7-hexahydro-s-indacen-4-yl)amino)-4H-1,2,4-triazole-3-carboxylate

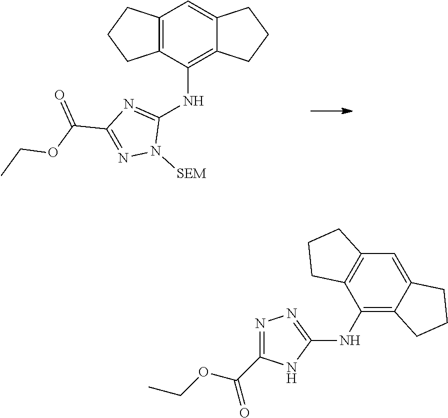 [SMILES: CCOC(=O)c1nc(Nc2c3c(cc4c2CCC4)CCC3)n(COCC[Si](C)(C)C)n1 ; CCOC(=O)c1nnc(Nc2c3c(cc4c2CCC4)CCC3)[nH]1]

Ethyl 5-((1,2,3,5,6,7-hexahydro-s-indacen-4-yl)amino)-1-((2-(trimethylsilyl)ethoxy)-methyl)-1H-1,2,4-triazole-3-carboxylate (Intermediate B1) (100 mg, 0.226 mmol) was dissolved in TFA (2 mL) and stirred at RT for 1 h. The reaction was concentrated in vacuo. The crude product was purified by acidic prep HPLC (50-80% MeOH in water) to afford the title compound (14 mg, 19% yield) as a flocculent white solid.

LCMS m/z 313.2 (M+H)+ (ES+); 311.0 (M−H)− (ES−)

¹H NMR (DMSO-d₆): δ 13.11 (s, 1H), 8.68 (s, 1H), 6.93 (s, 1H), 4.26 (q, J=7.1 Hz, 2H), 2.82 (t, J=7.4 Hz, 4H), 2.62 (t, J=7.3 Hz, 4H), 1.97 (p, J=7.4 Hz, 4H), 1.28 (t, J=7.1 Hz, 3H).

### Example 2: pyridin-3-ylmethyl 5-((1,2,3,5,6,7-hexahydro-s-indacen-4-yl)amino)-4H-1,2,4-triazole-3-carboxylate

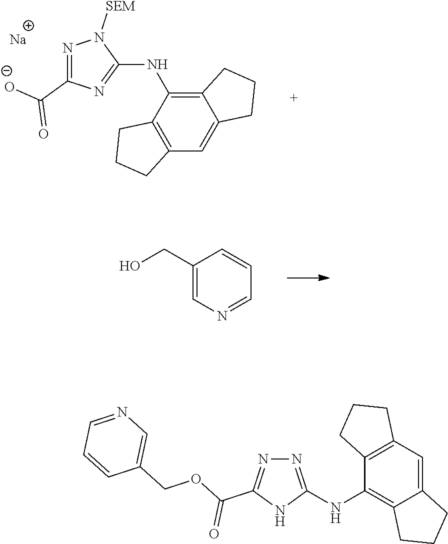 [SMILES: C[Si](C)(C)CCOCn1nc(C(=O)[O-])nc1Nc1c2c(cc3c1CCC3)CCC2 ; [Na+] ; OCc1cccnc1 ; O=C(OCc1cccnc1)c1nnc(Nc2c3c(cc4c2CCC4)CCC3)[nH]1]

The crude sodium 5-((1,2,3,5,6,7-hexahydro-s-indacen-4-yl)amino)-4H-1,2,4-triazole-3-carboxylate (Intermediate C1) (0.28 mmol) was dissolved in DMF (3 mL) and HATU (0.319 g, 0.840 mmol) was added, followed by pyridin-3-ylmethanol (82 μL, 0.84 mmol). The reaction was stirred at RT for 18 h and diluted with EtOAc (20 mL) and water (5 mL). The organic phases were washed with brine (2×5 mL) and the organics were dried (MgSO₄) and concentrated in vacuo. TFA (0.1 mL) was added to the residue and the reaction was stirred for 1 h, concentrated in vacuo and purified by acidic prep HPLC (35-65% MeOH in water) to afford the title compound (6 mg, 5% yield) as a white solid.

LCMS m/z 376.2 (M+H)⁺ (ES⁺).

¹H NMR (DMSO-d₆) δ 8.88 (s, 1H), 8.66 (d, J=2.2 Hz, 1H), 8.56 (dd, J=4.8, 1.7 Hz, NH), 7.89-7.82 (m, 1H), 7.43 (dd, J=7.8, 4.8 Hz, 1H), 6.91 (s, 7H), 5.33 (s, 2H), 2.81 (t, J=7.4 Hz, 4H), 2.62 (t, J=7.4 Hz, 4H), 1.95 (p, J=7.4 Hz, 4H). One exchangeable proton not observed.

The following examples were synthesised by methods analogous to those outlined above:

| Example | Structure | ¹H NMR | LCMS |
|---|---|---|---|
| 3 | 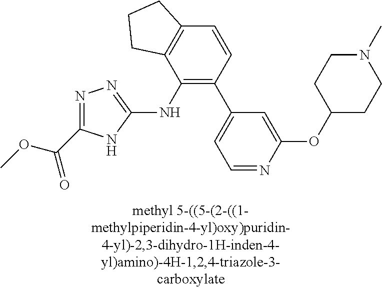 | ¹H NMR (DMSO-d₆) δ 8.68 (s, 1H), 8.07 (d, J = 5.3 Hz, 1H), 7.24 (d, J = 7.7 Hz, 1H), 7.17 (d, J = 7.7 Hz, 1H), 6.91 (dd, J = 5.3, 1.4 Hz, 1H), 6.77- 6.66 (m, 1H), 5.20-5.05 (m, 1H), 3.76 (s, 3H), 3.15 (app br s, 2H), 3.02-2.91 (m, 4H), 2.70-2.62 (m, 5H), 2.15-2.05 (m, 2H), 2.00 (p, J = 7.5 Hz, 2H), 1.85 (app br s, 2H). One exchangeable proton not observed. | m/z 449.3 (M + H)⁺ (ES⁺) |
| 4 | 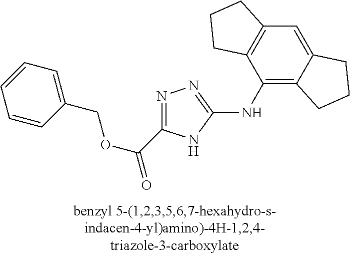 [SMILES: O=C(OCc1ccccc1)c1nnc(Nc2c3c(cc4c2CCC4)CCC3)[nH]1] | ¹H NMR (DMSO-d₆) δ 13.06 (s, 1H), 8.71 (s, 1H), 7.52-7.31 (m, 5H), 6.93 (s, 1H), 5.30 (s, 2H), 2.82 (t, J = 7.4 Hz, 4H), 2.62 (t, J = 7.3 Hz, 4H), 1.96 (p, J = 7.4 Hz, 4H). | m/z 375.2 (M + H)⁺ (ES⁺) 373.1 (M − H)⁻ (ES⁻) |
| 5 | 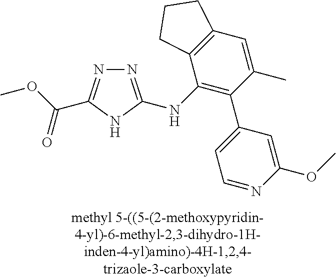 | ¹H NMR (DMSO-d₆) δ 8.31-8.21 (m, 1H), 8.11 (d, J = 5.2 Hz, 1H), 7.12 (s, 1H), 6.70 (dd, J = 5.2, 1.4 Hz, 1H), 6.54 (s, 1H), 3.82 (s, 3H), 3.75 (s, 3H), 2.90 (t, J = 7.4 Hz, 2H), 2.61-2.55 (m, 2H), 2.06- 1.93 (m, 5H). One exchangeable proton not observed. | m/z 380.2 (M + H)⁺ (ES⁺) |
| 6 | 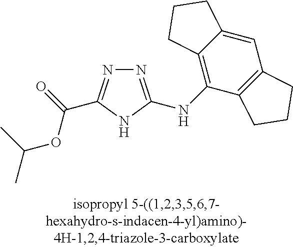 [SMILES: CC(C)OC(=O)c1nnc(Nc2c3c(cc4c2CCC4)CCC3)[nH]1] | ¹H NMR (DMSO-d₆) δ 13.07 (br s, 1H), 8.68 (br s, 1H), 6.93 (s, 1H), 5.08 (sept, J = 6.2 Hz, 1H), 2.81 (t, J = 7.4 Hz, 4H), 2.61 (t, J = 7.3 Hz, 4H), 1.96 (p, J = 7.4 Hz, 4H), 1.27 (d, J = 6.2 Hz, 6H). | m/z 327.2 (M + H)⁺ (ES⁺) 325.2 (M − H)⁻ (ES⁻) |
| 7 | 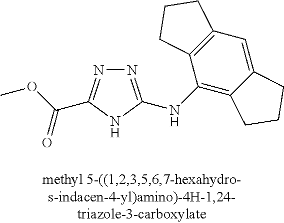 [SMILES: COC(=O)c1nnc(Nc2c3c(cc4c2CCC4)CCC3)[nH]1] | ¹H NMR (DMSO-d₆) δ 13.15 (br s, 1H), 8.68 (s, 1H), 6.93 (s, 1H), 3.78 (s, 3H), 2.82 (t, J = 7.3 Hz, 4H), 2.62 (t, J = 7.3 Hz, 4H), 1.96 (p, J = 7.4 Hz, 4H). | m/z 299.1 (M + H)⁺ (ES⁺) 297.0 (M − H)⁻ (ES⁻) |
| 8 | 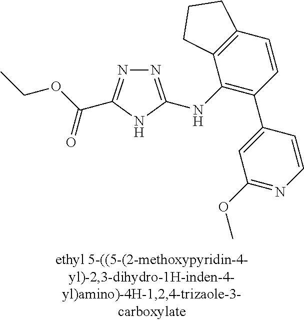 [SMILES: CCOC(=O)c1nnc(Nc2c(-c3ccnc(OC)c3)ccc3c2CCC3)[nH]1] | ¹H NMR (DMSO-d₆) δ 13.10 (br s, 1H), 8.67 (s, 1H), 8.09 (d, J = 5.3 Hz, 1H), 7.24 (d, J = 7.7 Hz, 1H), 7.19 (d, J = 7.7 Hz, 1H), 6.92 (dd, J = 5.3, 1.4 Hz, 1H), 6.73 (s, 1H), 4.23 (q, J = 7.1 Hz, 2H), 3.83 (s, 3H), 2.95 (t, J = 7.4 Hz, 2H), 2.66 (t, J = 7.4 Hz, 2H), 2.01 (p, J = 7.5 Hz, 2H), 1.26 (t, J = 7.1 Hz, 3H). | m/z 380.1 (M + H)⁺ (ES⁺) 378.1 (M − H)⁻ (ES⁻) |

### Examples—Biological Studies

NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 g/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hrs at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

96-Well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

| High | MCC950 (10 uM) | Compound 8-point half-log dilution |
|---|---|---|
| Low | Drug free control | |

The results of the pyroptosis assay are summarised in Table 1 below as THP IC₅₀.

Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP₃ inhibitory activity (≤2 μM = ‘+++’, ≤5 μM =
‘++’, ≤10 μM = ‘+’, not determined = ‘ND’).

| Example No | THP IC₅₀ | HWB IC₅₀ |
|---|---|---|
| 1 | +++ | ++ |
| 2 | + | ND |
| 3 | + | ND |
| 4 | ++ | ND |
| 5 | ++ | ND |
| 6 | + | ND |
| 7 | +++ | ND |
| 8 | ++ | ND |

As is evident from the results presented in Table 1, surprisingly in spite of the structural differences versus the prior art compounds, the compounds of the invention show high levels of NLRP3 inhibitory activity in the pyroptosis assay and in the human whole blood assay.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 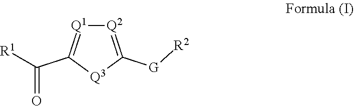
or a pharmaceutically acceptable salt or solvate thereof, wherein:
Q¹ and Q² are each independently selected from N or CR^{q}, provided that at least one of Q¹ and Q² is N;
Q³ is O, S or NR^{qq};
each R^{q} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
each R^{qq} is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
G is —O—, —C(R^{g})₂—, or —NR^{gg}—;
each R^{g} is independently selected from hydrogen or a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton, or any two R^{g} may, together with the carbon atom to which they are attached, form a cyclic group wherein the cyclic group may optionally be substituted; and
each R^{gg} is independently selected from hydrogen or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton;
R¹ is hydrogen, —OH, —NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position, wherein R² may optionally be further substituted.

2. The compound or pharmaceutically acceptable salt or solvate thereof, as claimed in claim 1, wherein:
(i) Q¹ and Q² are both N; and/or
(ii) Q³ is NR^{gg}, optionally wherein R^{qq} is independently selected from hydrogen or a C₁-C₄ alkyl or C₁-C₄ cycloalkyl group, wherein the C₁-C₄ alkyl or C₃-C₄ cycloalkyl group may optionally be substituted with one or more fluoro and/or chloro groups.

3. (canceled)

4. (canceled)

5. The compound or pharmaceutically acceptable salt or solvate thereof, as claimed in claim 1, wherein:
(i) Q³ is NH; and/or
(ii) G is —NH—.

6. (canceled)

7. The compound or pharmaceutically acceptable salt or solvate thereof, as claimed in, wherein:
(i) R¹ is a saturated or unsaturated C₁-C₂₀ hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms N, O or S in its carbon skeleton; or
(ii) R¹ is selected from R¹⁰—O—, (R¹⁰)NH— or (R¹⁰)₂N—, wherein each R¹⁰ is independently selected from a C₁-C₁₂ alkyl, C₂-C₁₂ alkenyl, C₂-C₁₂ alkynyl or R¹¹-L- group, wherein R¹¹ is a 2- to 12-membered cyclic group and L is a bond or a C₁-C₆ alkylene, C₂-C₆ alkenylene, or C₂-C₁₂ alkynylene group, or two R¹⁰ may, together with the nitrogen atom to which they are attached, form a 8- to 12-membered heterocyclic group, wherein any alkyl, alkenyl, alkynyl, alkylene, alkenylene or alkynylene group may optionally include one or more heteroatoms independently selected from oxygen and nitrogen in their carbon skeleton, and wherein any alkyl, alkenyl, alkynyl, alkylene, alkenylene, alkynylene, cyclic or heterocyclic group may optionally be substituted.

8. (canceled)

9. The compound or pharmaceutically acceptable salt or solvate thereof, as claimed in claim 1, wherein R¹ is R¹⁰—O—, wherein R¹⁰ is selected from a C₁-C₈ alkyl, C₂-C₈ alkenyl or R¹¹-L- group, wherein R¹¹ is a 3- to 7-membered monocyclic group or a 7- to 10-membered fused bicyclic group and L is a bond or a C₁-C₄ alkylene or C₂-C₄ alkenylene group, wherein any alkyl, alkenyl, alkylene or alkenylene group may optionally include one or two heteroatoms independently selected from oxygen and nitrogen in their carbon skeleton, wherein any alkyl, alkenyl, alkylene or alkenylene group may optionally be substituted with one or more substituents independently selected from halo, —CN, —OH, —NH₂ and oxo (═O), wherein any monocyclic or fused bicyclic group may optionally be substituted with one or more substituents independently selected from halo, —CN, —OH, —NH₂, oxo (═O), -Me, -Et, —OMe, —OEt, —NHMe, —NHEt, —N(Me)₂, —N(Me)Et or —N(Et)₂, and wherein any methyl (Me) or ethyl (Et) group may optionally be substituted with one or more halo groups.

10. The compound or pharmaceutically acceptable salt or solvate thereof, as claimed in claim 1, wherein:
R¹ is R¹⁰—O—, wherein R¹⁰ is selected from a C₁-C₆ alkyl, C₁-C₆ haloalkyl, C₃-C₆ cycloalkyl or C₃-C₆ halocycloalkyl group; or
R¹ is R¹¹—CH₂—O—, wherein R¹¹ is selected from a phenyl, halophenyl or 5- or 6-membered heteroaryl group, wherein the 5- or 6-membered heteroaryl group may optionally be halo-substituted.

11. The compound or pharmaceutically acceptable salt or solvate thereof, as claimed in claim 1, wherein R² is a phenyl or a 5- or 6-membered heteroaryl group, wherein the phenyl or the heteroaryl group is substituted at the α-position, or at the α and α′ positions, and wherein R² may optionally be further substituted.

12. (canceled)

13. The compound or pharmaceutically acceptable salt or solvate thereof, as claimed in claim 11, wherein:
(i) at least one substituent at the α and/or α′ positions comprises a carbon atom, or
(ii) both substituents at the α and α′ positions comprise a carbon atom.

14. (canceled)

15. The compound or pharmaceutically acceptable salt or solvate thereof, as claimed in claim 11, wherein —R² has a formula selected from: 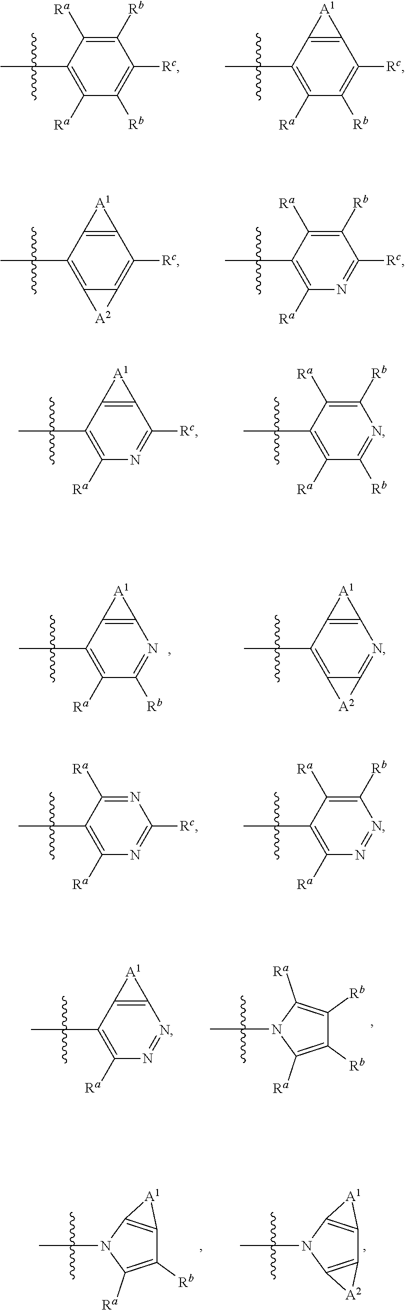 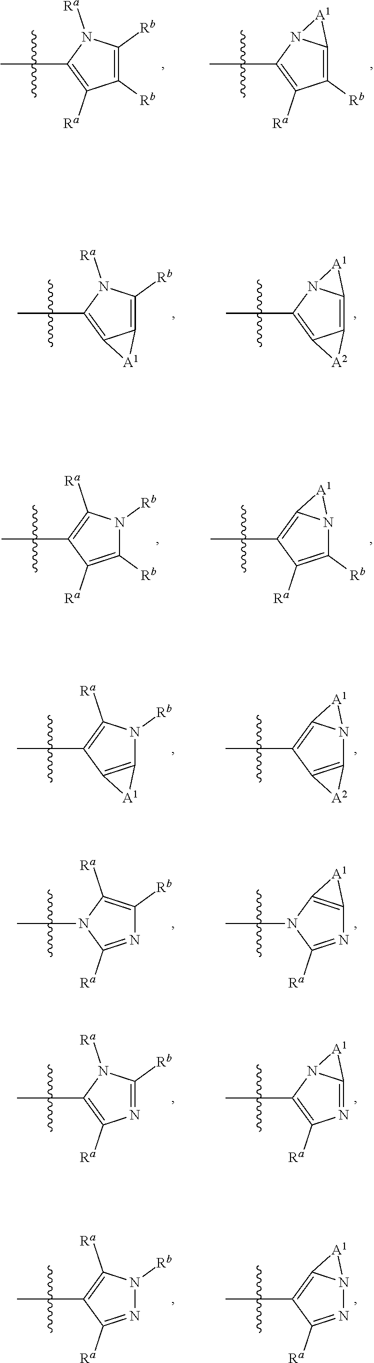 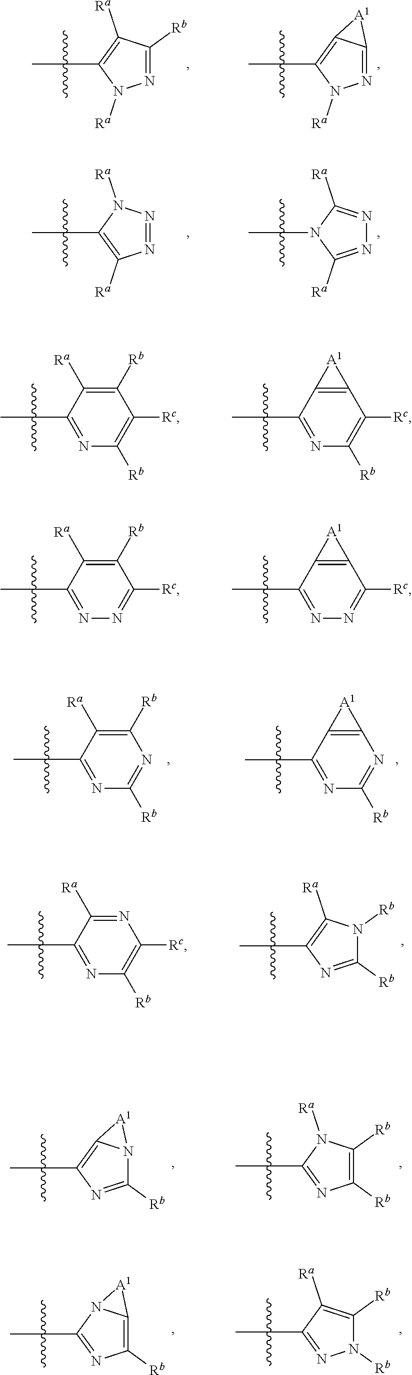 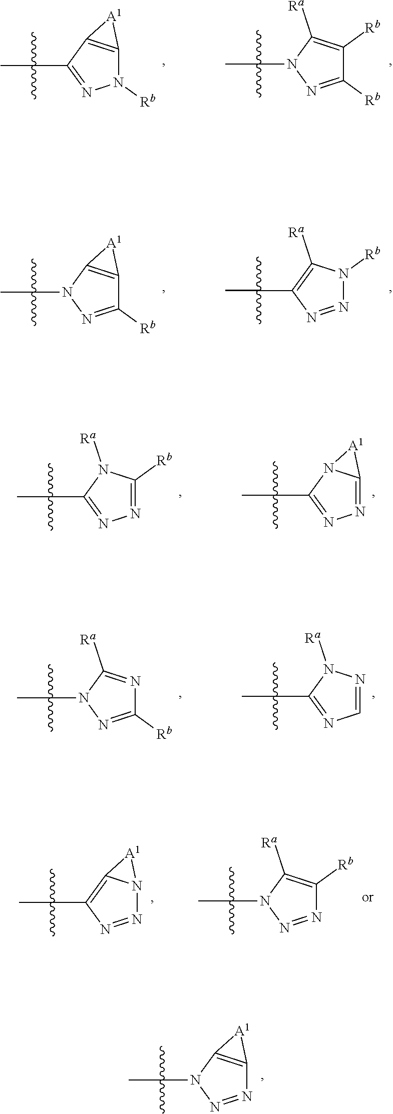
wherein:
A¹ and A² are each independently selected from a straight-chained alkylene group or a straight-chained alkenylene group, wherein one or two carbon atoms in the backbone of the alkylene or alkenylene group may optionally be replaced by one or two heteroatoms independently selected from nitrogen and oxygen, wherein any ring containing A¹ or A² is a 5- or 6-membered ring, and wherein the alkylene or alkenylene group may optionally be substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl);
each R^{a} is independently selected from hydrogen, halo, —R^{aa}, —OR^{aa} or —COR^{aa}, provided that at least one R^{a} is —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{aa} is independently selected from a C₁-C₆ alkyl, C₂-C₆ alkenyl, C₂-C₆ alkynyl or a 3- to 7-membered cyclic group, wherein each C₁-C₆ alkyl, C₂-C₆ alkenyl, or C₂-C₆ alkynyl group is optionally substituted with one or more substituents independently selected from halo, —OH, —CN, —NO₂, —O(C₁-C₄ alkyl) or —O(C₁-C₄ haloalkyl), and wherein each 3- to 7-membered cyclic group is optionally substituted with one or more substituents independently selected from halo, —OH, —NH₂, —CN, —NO₂, —B¹, —CH₂B¹, —OB¹, —OCH₂B¹, —NHB¹, —N(B¹)₂, —CONH₂, —CONHB¹, —C ON(B¹)₂, —NHCOB¹, —NB¹COB¹, or —B¹¹—;
each B¹ is independently selected from a C₁-C₆ alkyl, C₂-C₆ alkenyl, C₂-C₆ alkynyl, C₃-C₁₀ cycloalkyl, C₅-C₁₀ cycloalkenyl, C₆-C₁₀ aryl, or a 4- to 10-membered heterocyclic group containing one or two ring heteroatoms N and/or O, or two B¹ together with the nitrogen atom to which they are attached may form a 4- to 10-membered heterocyclic group containing one or two ring heteroatoms N and/or O, wherein any B¹ may optionally be halo-substituted and/or substituted with one or two substituents independently selected from —OH, —NH₂, —B¹², —OB¹², —NHB¹² or —N(B¹²)₂;
each B¹¹ is independently selected from a C₁-C₈ alkylene or C₂-C₅ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —B¹², —OB¹², —NHB¹² or —N(B¹²)₂;
each B¹² is independently selected from a C₁-C₃ alkyl or C₁-C₃ haloalkyl group;
each R^{c} is selected from hydrogen,
halo, —OH, —NO₂, —CN, —R^{cc}, —R^{cc}, —OR^{cc}, —COR^{cc}, —COOR^{cc}, —CONH₂, —CONHR^{cc}, —CON(R^{cc})₂, —C(═NH)R^{cc}, —C(═NH)NH₂, —C(═NH)NHR^{cc}, —C(═NH)N(R^{cc})₂, —C(═NR^{cc})R^{cc}, —C(═NR^{cc})NHR^{cc}, —C(═NR^{cc})N(R^{cc})₂, —C(═NOH)R^{cc} or —C(═NOR^{cc})R^{cc};
each R^{cc} is independently selected from C₁-C₄ alkyl, C₁-C₄ haloalkyl, C₃-C₄ cycloalkyl or C₃-C₄ halocycloalkyl, or any two R^{cc} attached to the same nitrogen atom may, together with the nitrogen atom to which they are attached, form a 3- to 6-membered saturated heterocyclic group, wherein the 3- to 6-membered saturated heterocyclic group is optionally halo substituted; and
each R^{cx} is selected from a 3- to 7-membered cyclic group, wherein the 3- to 7-membered cyclic group is optionally halo substituted.

16. The compound or pharmaceutically acceptable salt or solvate thereof, as claimed in claim 11, wherein R² contains from 10 to 35 atoms other than hydrogen or halogen.

17. The compound or pharmaceutically acceptable salt or solvate thereof, as claimed in claim 1, which is (a) a compound selected from the group consisting of: 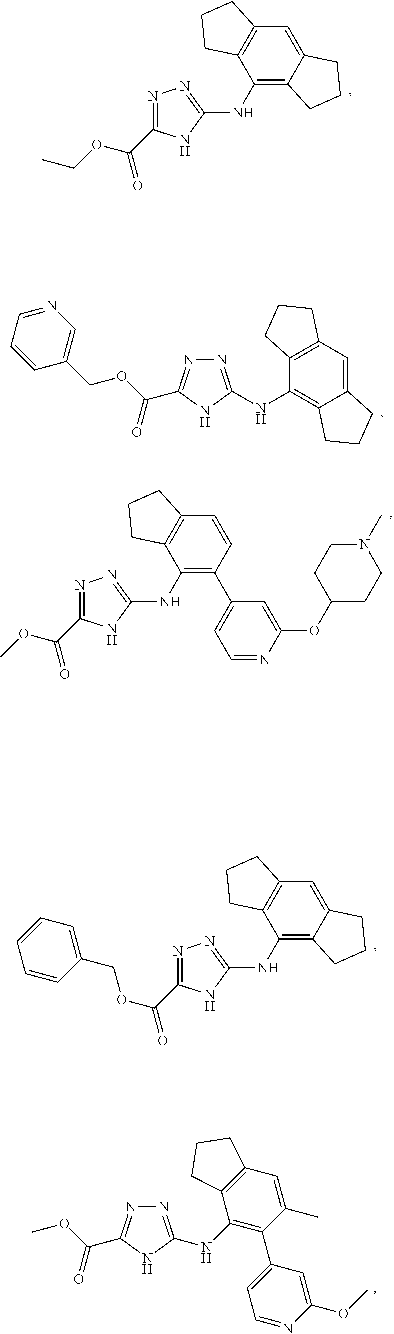 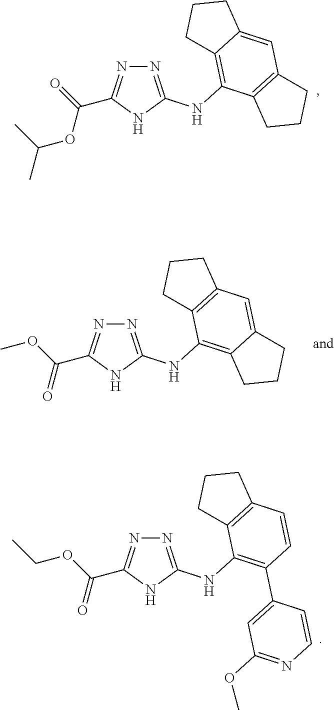 [SMILES: CC(C)OC(=O)c1nnc(Nc2c3c(cc4c2CCC4)CCC3)[nH]1 ; COC(=O)c1nnc(Nc2c3c(cc4c2CCC4)CCC3)[nH]1 ; CCOC(=O)c1nnc(Nc2c(-c3ccnc(OC)c3)ccc3c2CCC3)[nH]1]
or (b) a pharmaceutically acceptable salt or solvate of the selected compound.

18. A prodrug of the compound as claimed in claim 1, or a pharmaceutically acceptable salt or solvate thereof.

19. A pharmaceutical composition comprising the compound the pharmaceutically acceptable salt or solvate thereof, as claimed in claim 1, and a pharmaceutically acceptable excipient.

20. A method of treating or preventing a disease, disorder or condition in a subject, the method comprising the step of administering an effective amount of the compound or the pharmaceutically acceptable salt or solvate thereof, as claimed in claim 18, to the subject, thereby treating or preventing the disease, disorder or condition, optionally wherein the disease, disorder or condition is responsive to NLRP3 inhibition.

21. (canceled)

22. The method as claimed in claim 20, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) pain;
(xvii) a condition associated with diabetes;
(xviii) a condition associated with arthritis;
(xix) a headache;
(xx) a wound or burn; and
(xxi) any disease where an individual has been determined to carry a germline or somatic non-silent mutation in NLRP3.

23. The method as claimed in claim 20, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

24. A method of inhibiting NLRP3 in a subject, the method comprising administering the compound or the pharmaceutically acceptable salt or solvate thereof, as claimed in claim 1, to the subject thereby inhibiting NLRP3.

25. A method of analysing inhibition of NLRP3 or an effect of inhibition of NLRP3 by a compound, comprising contacting a cell or non-human animal with the compound or the pharmaceutically acceptable salt or solvate thereof, as claimed in claim 1, and analysing inhibition of NLRP3 or an effect of inhibition of NLRP3 in the cell or non-human animal by the compound.

26. The method as claimed in claim 20, wherein the compound or the pharmaceutically acceptable salt or solvate thereof is administered as a pharmaceutical composition further comprising a pharmaceutically acceptable excipient.

27. A method of treating or preventing a disease, disorder or condition in a subject, the method comprising the step of administering an effective amount of the prodrug or the pharmaceutically acceptable salt or solvate thereof, as claimed in claim 18, to the subject, thereby treating or preventing the disease, disorder or condition, optionally wherein the disease, disorder or condition is responsive to NLRP3 inhibition.
